(19) United States
(12) Patent Application Publication
(10) Pub. No.: US 2005/0118262 A1
(43) Pub. Date: Jun. 2, 2005

(54) Controlled release formulation

(76) Inventors: Jack Aurora, Lorraine (CA); Manmohan Singh Gill, Brampton (CA); Kenneth K. H. Ong, Scarborough (CA)

Correspondence Address: STERNE, KESSLER, GOLDSTEIN & FOX PLLC, 1100 NEW YORK AVENUE, N.W., WASHINGTON, DC, 20005, US

(21) Appl. No.: 10/943,147

(22) Filed: Sep. 17, 2004

Related U.S. Application Data
(60) Provisional application No. 60/503,584, filed on Sep. 17, 2003.

(51) Int. Cl. A61K 31/7076; A61K 9/22; A61K 35/78

(52) U.S. Cl. 424/468; 514/46; 424/731

(57) ABSTRACT

The present invention discloses a controlled release formulation suitable for oral administration comprising N⁶-cyclopentyl-5′-(N-ethyl)carboxamidoadenosine or a pharmaceutically acceptable salt or ester thereof incorporated in a controlled release matrix. The controlled release formulation of the invention is especially suitable for treating heart rhythm disturbances in a mammal.

This application claims the priority benefit under 35 U.S.C. § 119(e) of U.S. Provisional Application No. 60/503,584, filed Sep. 17, 2003, the entirety of which is incorporated by reference herein.

### BACKGROUND OF THE INVENTION

1. Field of the Invention

The present invention relates to a controlled release formulation for oral administration, processes for its preparation and to its medical use. In particular, the invention relates to a controlled release formulation comprising N⁶-cyclopentyl-5′-(N-ethyl)carboxamidoadenosine (selodenoson) or a pharmaceutically acceptable salt or ester thereof.

1. Related Art

In the mammalian heart, in order to circulate blood, contraction of the ventricles is initiated by an electrical impulse in the sinoatrial (SA) node that then passes through the atrioventricular (AV) node to the ventricles. Consistent rhythmic impulses produce a regular heart beat when the heart is functioning normally.

Various dysfunctions can occur in the heart to disturb the regular heart beat pattern and produce arrhythmias. For example, a condition of excessively rapid heart beats can occur that is defined as tachycardia. Tachycardia can originate from excessively rapid discharges from a malfunctioning SA node, electrical impulses passing through accessory pathways that prompt beats before they would normally occur, or re-entry of impulses in the AV node. In such cases, the disorder has been referred to as paroxsymal supraventricular tachycardia (PSVT), which in some patients can progress to congestive heart failure. Another heart rhythm disturbance, referred to as atrial fibrillation/flutter (AF), is characterized by a rapid and disordered beating of the atria, and is associated with an increased incidence of stroke. In atrial fibrillation, the heart beats at two to three times its normal rate, impairing the heart's ability to effectively pump blood to the rest of the body and causing symptoms ranging from shortness of breath to heart failure, potentially progressing to death. The above described heart rhythm disturbances can be classified as supraventricular tachyarrhythmias. Symptoms of these disorders can include discomfort due to palpitations, fatigue, dyspnea, as well as a sensation of lightheadedness and/or dizziness.

Approximately 6 million people worldwide experience atrial fibrillation. Although many treatments for atrial fibrillation are currently available, all cause significant side-effects ranging from blood pressure to a dangerously slow heart rate. Even adenosine-based treatments, the most popular treatments available, trigger side effects because they are not able to distinguish between the four types of adenosine receptors. The adenosine Al receptor is involved in atrial fibrillation.

N⁶-cyclopentyl-5′-(N-ethyl)carboxamidoadenosine (selodenoson) is a selective adenosine A₁ receptor agonist. International Patent Publication WO 01/37845 describes that N⁶-cyclopentyl-5 ′-(N-ethyl)carboxamidoadenosine possesses particularly desirable pharmacological properties useful in the treatment of heart rhythm disturbances. Such heart rhythm disturbances include supraventricular tachyarrhythmias, such as paroxysmal supraventricular tachycardia (PSVT) and atrial fibrillation/flutter (AF). WO 01/37845 describes parenteral, subcutaneous, intravenous, intramuscular, intraperitoneal, transdermal, buccal, or ocular routes of administration. Alternatively, or concurrently, administration can be by the oral route. WO 01/37845 describes only conventional oral dosage forms.

The selectivity of selodenoson to adenosine Al receptors provides for more effective dosing and fewer side-effects. However, a need in the art still exists for an oral controlled release formulation for the chronic management of heart rhythm disturbances, particularly atrial fibrillation. Such a formulation would enable patients to take the medication orally once or twice a day in order to increase compliance and maximize the therapeutic effect. Additionally, a controlled release formulation would enable relatively low plasma selodenoson concentrations for a longer period of time which would increase safety to the patient, as opposed to using high doses to increase the time the plasma drug levels are above the therapeutic concentration.

Controlled release formulations for oral administration containing N⁶-cyclopentyl-5′-(N-ethyl)carboxamidoadenosine (selodenoson) as active ingredient have not been previously described in the literature.

### SUMMARY OF THE INVENTION

The present invention provides an oral controlled release formulation containing N⁶-cyclopentyl-5′-(N-ethyl)carboxamidoadenosine (selodenoson) or a pharmaceutically acceptable salt or ester thereof suitable for administration at least once every twelve hours, e.g., up to once every twenty-four hours. The formulation is suitable for the treatment of heart rhythm disturbances, including atrial fibrillation or atrial flutter. Accordingly, the present invention provides an oral controlled release formulation, comprising N⁶-cyclopentyl-5′-(N-ethyl)carboxamidoadenosine (selodenoson) or a pharmaceutically acceptable salt or ester thereof and a pharmaceutically acceptable matrix adapted to provide a controlled release of N⁶-cyclopentyl-5′-(N-ethyl)carboxamidoadenosine or a pharmaceutically acceptable salt or ester thereof upon oral administration. The oral controlled release formulation of the invention is suitable for dosing every 12 hours or more.

The present invention provides an oral controlled release pharmaceutical formulation, comprising N⁶-cyclopentyl-5′-(N-ethyl)carboxamidoadenosine or a pharmaceutically acceptable salt or ester thereof and a pharmaceutically acceptable matrix adapted to provide a controlled release of N⁶-cyclopentyl-5′-(N-ethyl)carboxamidoadenosine (selodenoson) or a pharmaceutically acceptable salt or ester thereof upon oral administration, having a dissolution rate in vitro when measured using (i) Apparatus 3 described in the USP 23 using 300 mL reciprocating cylinder at 30 dips/minute in 250 mL 0.1 M hydrochloric acid from 0 minutes to 2 hours of dissolution and in 250 mL pH 6.8 phosphate buffer after 2 hours and up to 12 hours of dissolution, at 37.0±0.5° C. and using high performance liquid chromatography (HPLC) for quantification or (ii) Apparatus 2 (paddle) described in USP 23 at 50 rpm in 500 mL 0.1 M hydrochloric acid or 0.005% SDS-0.1 M hydrochloric acid from 0 minutes to 2 hours of dissolution and in 900 mL pH 6.8 phosphate buffer, or pH 6.8 phosphate buffer containing 0.5% SDS, after 2 hours and up to 12 hours of dissolution, at 37.0±0.5° C. and using high performance liquid chromatography (HPLC) for quantification,
between 0 and 35% selodenoson released after 15 minutes;
between 0 and 50% selodenoson released after 30 minutes;
between 0.5 and 60% selodenoson released after 45 minutes;
between 0.5 and 65% selodenoson released after 1 hour;
between 0.5 and 90% selodenoson released after 2 hours;
between 20 and 90% selodenoson released after 4 hours;
between 25 and 100% selodenoson released after 8 hours; and
greater than 30% selodenoson released after 12 hours, by weight.

Preferably, the above formulation has the following in vitro release rate:
between 0 and 35% selodenoson released after 15 minutes;
between 0.5 and 50% selodenoson released after 30 minutes;
between 0.5 and 60% selodenoson released after 45 minutes;
between 1 and 65% selodenoson released after 1 hour;
between 2 and 85% selodenoson released after 2 hours;
between 25 and 90% selodenoson released after 4 hours;
between 40 and 100% selodenoson released after 8 hours; and
greater than 55% selodenoson released after 12 hours, by weight.

Further, the present invention provides an oral controlled release pharmaceutical formulation, comprising N⁶-cyclopentyl-5′-(N-ethyl)carboxamidoadenosine or a pharmaceutically acceptable salt or ester thereof and a pharmaceutically acceptable matrix adapted to provide a controlled release of N⁶-cyclopentyl-5′-(N-ethyl)carboxamidoadenosine (selodenoson) or a pharmaceutically acceptable salt or ester thereof upon oral administration, having a dissolution rate in vitro when measured using (i) Apparatus 3 described in the USP 23 using 300 mL reciprocating cylinder at 30 dips/minute in 250 mL 0.1 M hydrochloric acid from 0 minutes to 2 hours of dissolution and in 250 mL pH 6.8 phosphate buffer after 2 hours and up to 12 hours of dissolution, at 37.0±0.5° C. and using high performance liquid chromatography (HPLC) for quantification or (ii) Apparatus 2 (paddle) described in USP 23 at 50 rpm in 500 mL 0.1 M hydrochloric acid or 0.005% SDS-0.1 M hydrochloric acid from 0 minutes to 2 hours of dissolution and in 900 mL pH 6.8 phosphate buffer, or pH 6.8 phosphate buffer containing 0.5% SDS, after 2 hours and up to 12 hours of dissolution, at 37.0±0.5° C. and using high performance liquid chromatography (HPLC) for quantification,
between 0 and 35% selodenoson released after 15 minutes;
between 5 and 50% selodenoson released after 30 minutes;
between 10 and 60% selodenoson released after 45 minutes;
between 10 and 65% selodenoson released after 1 hour;
between 15 and 90% selodenoson released after 2 hours;
between 20 and 90% selodenoson released after 4 hours;
between 25 and 100% selodenoson released after 8 hours; and
greater than 30% selodenoson released after 12 hours, by weight.

Preferably, the above formulation has the following in vitro release rate:
between 0 and 35% selodenoson released after 15 minutes;
between 5 and 50% selodenoson released after 30 minutes;
between 10 and 60% selodenoson released after 45 minutes;
between 15 and 65% selodenoson released after 1 hour;
between 25 and 85% selodenoson released after 2 hours;
between 35 and 90% selodenoson released after 4 hours;
between 45 and 100% selodenoson released after 8 hours; and
greater than 55% selodenoson released after 12 hours, by weight.

Further, the present invention provides a controlled release oral formulation suitable for dosing every 12 hours or more, comprising
a substrate comprising a pharmaceutically effective amount of N⁶-cyclopentyl-5′-(N-ethyl)carboxamidoadenosine or a pharmaceutically acceptable salt or ester thereof; and
said substrate being incorporated in a controlled release matrix;
said formulation having a dissolution rate in vitro when measured using (i) Apparatus 3 described in the USP 23 using 300 mL reciprocating cylinder at 30 dips/minute in 250 mL 0.1 M hydrochloric acid from 0 minutes to 2 hours of dissolution and in 250 mL pH 6.8 phosphate buffer after 2 hours and up to 12 hours of dissolution, at 37.0±0.5° C. and using high performance liquid chromatography (HPLC) for quantification or (ii) Apparatus 2 (paddle) described in USP 23 at 50 rpm in 500 mL 0.1 M hydrochloric acid, or 0.05% SDS-0. 1 M hydrochloric acid, from 0 minutes to 2 hours of dissolution and in 900 mL pH 6.8 phosphate buffer, or pH 6.8 phosphate buffer containing 0.5% SDS, after 2 hours and up to 12 hours of dissolution, at 37.0±0.5° C. and using high performance liquid chromatography (HPLC) for quantification, between 0 and 35% selodenoson released after 15 minutes;
between 0 and 50% selodenoson released after 30 minutes;
between 0.5 and 60% selodenoson released after 45 minutes;
between 0.5 and 65% selodenoson released after 1 hour;
between 0.5 and 90% selodenoson released after 2 hours;
between 20 and 90% selodenoson released after 4 hours;
between 25 and 100% selodenoson released after 8 hours; and
greater than 30% selodenoson released after 12 hours, by weight.

The formulations of the present invention provide a therapeutic effect for at least 12 hours, preferably for about 24 hours after oral administration. Preferably, the above formulation has a dissolution rate in vitro when measured using (i) Apparatus 3 described in the USP 23 using 300 mL reciprocating cylinder at 30 dips/minute in 250 mL 0.1 M hydrochloric acid from 0 minutes to 2 hours of dissolution and in 250 mL pH 6.8 phosphate buffer after 2 hours and up to 12 hours of dissolution, at 37.0±0.5° C. and using high performance liquid chromatography (HPLC) for quantification or (ii) Apparatus 2 (paddle) described in USP 23 at 50 rpm in 500 mL 0.1 M hydrochloric acid or 0.005% SDS-0.1 M hydrochloric acid from 0 minutes to 2 hours of dissolution and in 900 mL pH 6.8 phosphate buffer, or pH 6.8 phosphate buffer containing 0.5% SDS, after 2 hours and up to 12 hours of dissolution, at 37.0±0.5° C. and using high performance liquid chromatography (HPLC) for quantification,
between 0 and 35% selodenoson released after 15 minutes;
between 0.5 and 50% selodenoson released after 30 minutes;
between 0.5 and 60% selodenoson released after 45 minutes;
between 1 and 65% selodenoson released after 1 hour;
between 2 and 85% selodenoson released after 2 hours;
between 25 and 90% selodenoson released after 4 hours;
between 40 and 100% selodenoson released after 8 hours; and
greater than 55% selodenoson released after 12 hours, by weight.

In one aspect, the present invention provides a controlled release oral formulation suitable for dosing every 12 hours or more, comprising
a substrate comprising a pharmaceutically effective amount of N⁶-cyclopentyl-5′-(N-ethyl)carboxamidoadenosine or a pharmaceutically acceptable salt or ester thereof; and
said substrate being incorporated in a controlled release matrix;
said formulation having a dissolution rate in vitro when measured using (i) Apparatus 3 described in the USP 23 using 300 mL reciprocating cylinder at 30 dips/minute in 250 mL 0.1 M hydrochloric acid from 0 minutes to 2 hours of dissolution and in 250 mL pH 6.8 phosphate buffer after 2 hours and up to 12 hours of dissolution, at 37.0±0.5° C. and using high performance liquid chromatography (HPLC) for quantification or (ii) Apparatus 2 (paddle) described in USP 23 at 50 rpm in 500 mL 0.1 M hydrochloric acid, or 0.05% SDS-0. 1 M hydrochloric acid, from 0 minutes to 2 hours of dissolution and in 900 mL pH 6.8 phosphate buffer, or pH 6.8 phosphate buffer containing 0.5% SDS, after 2 hours and up to 12 hours of dissolution, at 37.0±0.5° C. and using high performance liquid chromatography (HPLC) for quantification,
between 0 and 35% selodenoson released after 15 minutes;
between 5 and 50% selodenoson released after 30 minutes;
between 10 and 60% selodenoson released after 45 minutes;
between 10 and 65% selodenoson released after 1 hour;
between 15 and 90% selodenoson released after 2 hours;
between 20 and 90% selodenoson released after 4 hours;
between 25 and 100% selodenoson released after 8 hours; and
greater than 30% selodenoson released after 12 hours, by weight.

Preferably, the above formulation has a dissolution rate in vitro when measured using (i) Apparatus 3 described in the USP 23 using 300 mL reciprocating cylinder at 30 dips/minute in 250 mL 0.1 M hydrochloric acid from 0 minutes to 2 hours of dissolution and in 250 mL pH 6.8 phosphate buffer after 2 hours and up to 12 hours of dissolution, at 37.0±0.5° C. and using high performance liquid chromatography (HPLC) for quantification or (ii) Apparatus 2 (paddle) described in USP 23 at 50 rpm in 500 mL 0.1 M hydrochloric acid or 0.005% SDS-0. 1 M hydrochloric acid from 0 minutes to 2 hours of dissolution and in 900 mL pH 6.8 phosphate buffer, or pH 6.8 phosphate buffer containing 0.5% SDS, after 2 hours and up to 12 hours of dissolution, at 37.0±0.5° C. and using high performance liquid chromatography (HPLC) for quantification,
between 0 and 35% selodenoson released after 15 minutes;
between 5 and 50% selodenoson released after 30 minutes;
between 10 and 60% selodenoson released after 45 minutes;
between 15 and 65% selodenoson released after 1 hour;
between 25 and 85% selodenoson released after 2 hours;
between 35 and 90% selodenoson released after 4 hours;
between 45 and 100% selodenoson released after 8 hours; and
greater than 55% selodenoson released after 12 hours, by weight.

The present invention also provides a solid, controlled release oral formulation, comprising a pharmaceutically effective amount of N⁶-cyclopentyl-5′-(N-ethyl)carboxamidoadenosine or a pharmaceutically acceptable salt or ester thereof and a controlled release matrix.

Further, the present invention provides a process for preparing a solid, controlled release oral formulation, comprising incorporating a pharmaceutically effective amount of N⁶-cyclopentyl-5′-(N-ethyl)carboxamidoadenosine or a pharmaceutically acceptable salt or ester thereof in a controlled release matrix such that said formulation as a dosage form provides a therapeutic effect for at least 12 hours after oral administration.

The present invention is also directed to the use of N⁶-cyclopentyl-5′-(N-ethyl)carboxamidoadenosine or a pharmaceutically acceptable salt thereof in the manufacture of a oral controlled release formulation for treating heart rhythm disturbances in a mammal.

Additional embodiments and advantages of the invention will be set forth in part in the description that follows, and in part, will be obvious from the description, or may be learned by practice of the invention. The embodiments and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

### BRIEF DESCRIPTION OF THE DRAWINGS

FIG. 1 depicts graphically the results of the dissolution tests described in Example 3 for Tablet 1, 2, 3, and 4 as a percent selodenoson released as a function of time.

FIG. 2 depicts graphically the results of the dissolution tests described in Example 5 for Capsule 1, 2, and 3 as a percent selodenoson released as a function of time.

FIG. 3 depicts graphically the results of the dissolution test described in Example 9 for Tablet 5 as a percent selodenoson released as a function of time.

FIG. 4 depicts graphically the results of the dissolution test described in Example 9 for Tablet 6 as a percent selodenoson released as a function of time.

FIG. 5 depicts graphically the results of the dissolution test described in Example 9 for Tablet 7 as a percent selodenoson released as a function of time.

FIG. 6 depicts graphically the results of the dissolution test described in Example 10 for Capsule 4 as a percent selodenoson released as a function of time.

FIG. 7 depicts graphically the results of the dissolution test described in Example 10 for Capsule 5 as a percent selodenoson released as a function of time.

### DETAILED DESCRIPTION OF THE INVENTION

N⁶-cyclopentyl-5′-(N-ethyl)carboxamidoadenosine (hereinafter also “selodenoson”) has the following chemical formula:

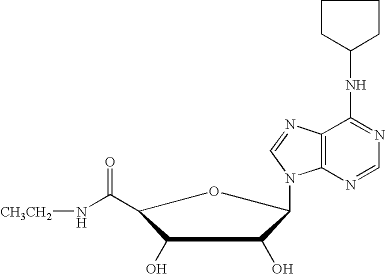

This compound is also known as 1-[6-(cyclopentylamino)-9H-purin-9-yl]-1-deoxy-N-ethyl-β-D-ribofuranuronamide, N-⁵′-ethyl-N⁶-(cyclopentyl)ade-nosine-5′-uronamide, or N⁶-cyclopentyladenosine-5′-ethylcarboxamide.

Suitable pharmaceutically acceptable salts of selodenoson for use according to the present invention include the conventional non-toxic salts or the quaternary ammonium salts which are formed, e.g., from inorganic or organic acids or bases. Examples of such acid addition salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, ftumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, nitrate, oxalate, pamoate, pectinate, persulfate, 3-phenylpropionate, picrate, pivalate, propionate, succinate, sulfate, tartrate, thiocyanate, tosylate, and undecanoate. Also, the basic nitrogen-containing groups may be quaternized with such agents as lower alkyl halides, such as methyl, ethyl, propyl, and butyl chloride, bromides and iodides; dialkyl sulfates like dimethyl, diethyl, dibutyl; and diamyl sulfates, long chain halides such as decyl, lauryl, myristyl and stearyl chlorides, bromides and iodides, aralkyl halides like benzyl and phenethyl bromides and others.

Suitable pharmaceutically acceptable esters of selodenoson for use according to the present invention include organic acid esters of the hydroxyl groups at the 2′ and 3′ positions of the ribofuranose moiety. Ester groups are preferably of the type which are relatively, readily hydrolyzed under physiological conditions. Useful esters include those having an acyl group selected from the group consisting of acetyl, propionyl, butyryl and benzoyl groups.

By “controlled release” it is meant for purposes of the present invention that pharmaceutically active N⁶-cyclopentyl-5′-(N-ethyl)carboxamidoadenosine is released from the formulation at a controlled rate such that therapeutically beneficial blood levels of the pharmaceutically active compound are maintained over an extended period of time, e.g., providing a 12 hour or a 24 hour dosage form.

N⁶-cyclopentyl-5′-(N-ethyl)carboxamidoadenosine (selodenoson) is soluble in water and highly soluble in water under acidic conditions. This water solubility makes the modulation of the rate of the dissolution of selodenoson in a controlled release fashion very difficult.

It has been found that in order to allow for controlled release of N⁶-cyclopentyl-5′-(N-ethyl)carboxamidoadenosine over at least a 12-hour period following oral administration, the in vitro release rate desirably corresponds to the following rate of N⁶-cyclopentyl-5′-(N-ethyl)carboxamidoadenosine released, measured using (i) Apparatus 3 described in the USP 23 using 300 mL reciprocating cylinder at 30 dips/minute in 250 mL 0.1 M hydrochloric acid from 0 minutes to 2 hours of dissolution and in 250 mL pH 6.8 phosphate buffer after 2 hours and up to 12 hours of dissolution, at 37.0±0.5° C. and using high performance liquid chromatography (HPLC) for quantification or (ii) Apparatus 2 (paddle) described in USP 23 at 50 rpm in 500 mL 0.1 M hydrochloric acid or 0.005% SDS-0. 1 M hydrochloric acid from 0 minutes to 2 hours of dissolution and in 900 mL pH 6.8 phosphate buffer, or pH 6.8 phosphate buffer containing 0.5% SDS, after 2 hours and up to 12 hours of dissolution, at 37.0±0.5° C. and using high performance liquid chromatography (HPLC) for quantification:
between 0 and 35% selodenoson released after 15 minutes;
between 0 and 50% selodenoson released after 30 minutes;
between 0.5 and 60% selodenoson released after 45 minutes;
between 0.5 and 65% selodenoson released after 1 hour;
between 0.5 and 90% selodenoson released after 2 hours;
between 20 and 90% selodenoson released after 4 hours;
between 25 and 100% selodenoson released after 8 hours; and
greater than 30% selodenoson released after 12 hours, by weight.

Preferably, the in vitro release rate corresponds to the following rate of N⁶-cyclopentyl-5′-(N-ethyl)carboxamidoadenosine released, measured using Apparatus 3 or Apparatus 2 as described above:
between 0 and 35% selodenoson released after 15 minutes;
between 0.5 and 50% selodenoson released after 30 minutes;
between 0.5 and 60% selodenoson released after 45 minutes;
between 1 and 65% selodenoson released after 1 hour;
between 2 and 85% selodenoson released after 2 hours;
between 25 and 90% selodenoson released after 4 hours;
between 40 and 100% selodenoson released after 8 hours; and
greater than 55% selodenoson released after 12 hours, by weight.

In one aspect, in order to allow for controlled release of N⁶-cyclopentyl-5′-(N-ethyl)carboxamidoadenosine over at least a 12-hour period following oral administration, the in vitro release rate corresponds to the following rate of N⁶-cyclopentyl-5′-(N-ethyl)carboxamidoadenosine released, measured using (i) Apparatus 3 described in the USP 23 using 300 mL reciprocating cylinder at 30 dips/minute in 250 mL 0.1 M hydrochloric acid from 0 minutes to 2 hours of dissolution and in 250 mL pH 6.8 phosphate buffer after 2 hours and up to 12 hours of dissolution, at 37.0±0.5° C. and using high performance liquid chromatography (HPLC) for quantification or (ii) Apparatus 2 (paddle) described in USP 23 at 50 rpm in 500 mL 0.1 M hydrochloric acid or 0.005% SDS-0.1 M hydrochloric acid from 0 minutes to 2 hours of dissolution and in 900 mL pH 6.8 phosphate buffer, or pH 6.8 phosphate buffer containing 0.5% SDS, after 2 hours and up to 12 hours of dissolution, at 37.0±0.5° C. and using high performance liquid chromatography (HPLC) for quantification:
between 0 and 35% selodenoson released after 15 minutes;
between 5 and 50% selodenoson released after 30 minutes;
between 10 and 60% selodenoson released after 45 minutes;
between 10 and 65% selodenoson released after 1 hour;
between 15 and 90% selodenoson released after 2 hours;
between 20 and 90% selodenoson released after 4 hours;
between 25 and 100% selodenoson released after 8 hours; and
greater than 30% selodenoson released after 12 hours, by weight.

Preferably, the in vitro release rate corresponds to the following rate of N⁶-cyclopentyl-5′-(N-ethyl)carboxamidoadenosine released, measured using Apparatus 3 or Apparatus 2 as described above:

TABLE 1

| | % selodenoson |
|---|---|
| Time/min. | released |
| 15 | 0-35 |
| 30 | 5-50 |
| 45 | 10-60 |
| 60 | 15-65 |
| 120 | 25-85 |
| 240 | 35-90 |
| 480 | 45-100 |
| 720 | >55 |

A suitable formulation suited for once-a-day dosing has an in vitro release rate corresponding to the following rate of selodenoson released, measured using Apparatus 3 as described above:

TABLE 2

| | % selodenoson |
|---|---|
| Time/min. | released |
| 15 | 0-20 |
| 30 | 5-30 |
| 45 | 10-40 |
| 60 | 10-45 |
| 120 | 15-60 |
| 240 | 20-65 |
| 480 | 25-85 |
| 720 | 30-100 |

Another suitable formulation suited for once-a-day dosing has an in vitro release rate corresponding to the following rate of selodenoson released, measured using Apparatus 3 as described above:

TABLE 3

| | % selodenoson |
|---|---|
| Time/min. | released |
| 15 | 0-20 |
| 30 | 10-30 |
| 45 | 15-40 |
| 60 | 20-45 |
| 120 | 30-60 |
| 240 | 40-65 |
| 480 | 45-85 |
| 720 | 60-100 |

Preferably, the formulations having the release rates described in Table 2 and Table 3 above are in the form of hydrophobic tablets.

A formulation particularly suitable for once-a-day dosing has an in vitro release rate corresponding to the following rate of selodenoson released, measured using Apparatus 3 as described above:

TABLE 4

| | % selodenoson |
|---|---|
| Time/min. | released |
| 15 | 0-15 |
| 30 | 5-20 |
| 45 | 10-25 |
| 60 | 15-30 |
| 120 | 25-45 |
| 240 | 35-55 |
| 480 | 50-70 |
| 720 | 70-95 |

Preferably, the formulation having the release rate described above in Table 4 is in the form of a hydrophilic tablet.

Further, a suitable controlled release formulation in accordance of the present invention suitable for once-a-day dosing has an in vitro release rate corresponding to the following rate of selodenoson released, measured using Apparatus 2 as described above:

TABLE 5

| | % selodenoson |
|---|---|
| Time/min. | released |
| 15 | 5-35 |
| 30 | 10-50 |
| 45 | 20-60 |
| 60 | 25-65 |
| 120 | 35-90 |
| 240 | 55-90 |
| 480 | 60-100 |
| 720 | 60-100 |

Preferably, the formulation having the above release rate is in the form of a capsule fill with controlled release spheroids (pellets).

Another advantageous dissolution rate in vitro upon release of the controlled release formulation for administration once daily according to the invention is between 5 and 20% by weight selodenoson released after 15 minutes, between 10 and 30% by weight selodenoson released after 30 minutes, between 15 and 35% by weight selodenoson released after 45 minutes, between 20 and 40% by weight selodenoson released after 1 hour, between 30 and 55% by weight selodenoson released after 2 hours, between 40 and 65% by weight selodenoson released after 4 hours, between 45 and 75% by weight selodenoson released after 8 hours, and between 60 and 85% by weight selodenoson released after 12 hours, measured using Apparatus 3 as described above. Preferably, the formulation having the above release rate is in the form of a hydrophobic tablet. Suitably, the hydrophobic tablet contains about 3 mg of selodenoson or a pharmaceutically acceptable salt or ester thereof.

Advantageously, a controlled release formulation according to the present invention, which is a hydrophobic tablet containing selodenoson, has the following dissolution rate in vitro measured using Apparatus 3 as described above: between 10 and 20% by weight selodenoson released after 15 minutes, between 15 and 30% by weight selodenoson released after 30 minutes, between 20 and 40% by weight selodenoson released after 45 minutes, between 25 and 45% by weight selodenoson released after 1 hour, between 40 and 60% by weight selodenoson released after 2 hours, between 45 and 65% by weight selodenoson released after 4 hours, between 60 and 80% by weight selodenoson released after 8 hours, and between 75 and 90% by weight selodenoson released after 12 hours. Suitably the hydrophobic tablet contains about 1 mg of selodenoson or a pharmaceutically acceptable salt or ester thereof.

A further advantageous dissolution rate in vitro upon release of the controlled release formulation for administration once daily according to the invention is between 5 and 15% by weight selodenoson released after 15 minutes, between 10 and 20% by weight selodenoson released after 30 minutes, between 15 and 30% by weight selodenoson released after 45 minutes, between 15 and 30% by weight selodenoson released after 1 hour, between 30 and 45% by weight selodenoson released after 2 hours, between 40 and 55% by weight selodenoson released after 4 hours, between 55 and 70% by weight selodenoson released after 8 hours, and between 70 and 95% by weight selodenoson released after 12 hours, measured using Apparatus 3 as described above. Preferably, the formulation having the above release rate is in the form of a hydrophilic tablet. Suitably the hydrophilic tablet contains about 3 mg of selodenoson or a pharmaceutically acceptable salt or ester thereof.

Another advantageous dissolution rate in vitro upon release of the controlled release formulation for administration once daily according to the invention is between 0 and 15% by weight selodenoson released after 15 minutes, between 5 and 20% by weight selodenoson released after 30 minutes, between 10 and 20% by weight selodenoson released after 45 minutes, between 15 and 25% by weight selodenoson released after 1 hour, between 25 and 40% by weight selodenoson released after 2 hours, between 35 and 50% by weight selodenoson released after 4 hours, between 50 and 65% by weight selodenoson released after 8 hours, and between 75 and 90% by weight selodenoson released after 12 hours, measured using Apparatus 3 as described above. Preferably, the formulation having the above release rate is in the form of a hydrophilic tablet. Suitably the hydrophilic tablet contains about 1 mg of selodenoson or a pharmaceutically acceptable salt or ester thereof.

Further, a suitable dissolution rate in vitro upon release of the controlled release formulation for administration once daily according to the invention is between 10 and 35% by weight selodenoson released after 15 minutes, between 20 and 50% by weight selodenoson released after 30 minutes, between 25 and 60% by weight selodenoson released after 45 minutes, between 30 and 65% by weight selodenoson released after 1 hour, between 45 and 90% by weight selodenoson released after 2 hours, between 55 and 90% by weight selodenoson released after 4 hours, between 60 and 95% by weight selodenoson released after 8 hours, and between 65 and 100% by weight selodenoson released after 12 hours, measured using Apparatus 2 as described above in 500 mL 0.1 M hydrochloric acid from 0 minutes to 2 hours of dissolution and in 900 mL pH 6.8 phosphate buffer after 2 hours and up to 12 hours of dissolution. Preferably, the formulation having the above release rate is in the form of a capsule filled with controlled release spheroids (pellets).

Advantageously, a controlled release formulation according to the present invention has the following dissolution rate in vitro measured using Apparatus 2 as described above in 500 mL 0.005% SDS-0.1 M hydrochloric acid from 0 minutes to 2 hours of dissolution and in 900 mL pH 6.8 phosphate buffer containing 0.5% SDS after 2 hours and up to 12 hours of dissolution: between 5 and 20% by weight selodenoson released after 15 minutes, between 15 and 30% by weight selodenoson released after 30 minutes, between 20 and 40% by weight selodenoson released after 45 minutes, between 25 and 50% by weight selodenoson released after 1 hour, between 35 and 70% by weight selodenoson released after 2 hours, between 60 and 90% by weight selodenoson released after 4 hours, between 70 and 100% by weight selodenoson released after 8 hours, and between 80 and 100% by weight selodenoson released after 12 hours. This formulation is preferably a capsule filled with controlled release pellets (spheroids) containing selodenoson.

Advantageously, a controlled release formulation according to the present invention has the following dissolution rate in vitro measured using Apparatus 2 as described above in 500 mL 0.005% SDS-0.1 M hydrochloric acid from 0 minutes to 2 hours of dissolution and in 900 mL pH 6.8 phosphate buffer containing 0.5% SDS after 2 hours and up to 12 hours of dissolution: between 0 and 5% by weight selodenoson released after 15 minutes, between 0 and 5% by weight selodenoson released after 30 minutes, between 0.5 and 6% by weight selodenoson released after 45 minutes, between 0.5 and 6% by weight selodenoson released after 1 hour, between 0.5 and 20% by weight selodenoson released after 2 hours, between 20 and 65% by weight selodenoson released after 4 hours, between 30 and 80% by weight selodenoson released after 8 hours, and between 50 and 100% by weight selodenoson released after 12 hours. Preferably, the dissolution rate in vitro measured as described above is as follows: between 0 and 3% by weight selodenoson released after 15 minutes, between 0.5 and 3% by weight selodenoson released after 30 minutes, between 0.5 and 5% by weight selodenoson released after 45 minutes, between 1 and 5% by weight selodenoson released after 1 hour, between 2 and 15% by weight selodenoson released after 2 hours, between 25 and 60% by weight selodenoson released after 4 hours, between 40 and 75% by weight selodenoson released after 8 hours, and between 60 and 90% by weight selodenoson released after 12 hours. This formulation is preferably a capsule filled with controlled release pellets (spheroids) containing selodenoson coated with a controlled release film coat.

The in vitro release rates mentioned herein are, except where otherwise specified, those obtained by measurement using (i) Apparatus 3, i.e., reciprocating cylinder, as described in the Unites States Pharmacopeia (USP) 23 (p. 1793-1794, United States Pharmacopeial Convention, Inc. (1995)) in 300 mL reciprocating cylinder at 30 dips/minute in 250 mL 0.1 M hydrochloric acid at 0 minutes to 2 hours of dissolution and in 250 mL pH 6.8 phosphate buffer at 2 to 12 hours of dissolution, at 37.0±0.5° C. and using high performance liquid chromatography (HPLC) for quantification or (ii) Apparatus 2, i.e., the paddle method, as described in the Unites States Pharmacopeia (USP) 23 (p. 1791-1793, United States Pharmacopeial Convention, Inc. (1995)) at 50 rpm in 500 mL 0.1 M hydrochloric acid, or 0.005% SDS-0.1 M hydrochloric acid, from 0 minutes to 2 hours of dissolution and in 900 mL pH 6.8 phosphate buffer, or pH 6.8 phosphate buffer containing 0.5% SDS, after 2 hours and up to 12 hours of dissolution, at 37.0±0.5° C. and using high performance liquid chromatography (HPLC) for quantification. The in vitro release rates of controlled release formulations according to the present invention in the form of a tablet have been measured using Apparatus 3 and those in the form of multiparticulates have been measured using Apparatus 2.

The controlled release formulation according to the present invention can be used for treating heart rhythm disturbances in mammals. The treatment of a heart rhythm disturbance episode and optional prophylactic maintenance therapy to minimize the further occurrence of heart rhythm disturbances are both contemplated as useful aspects of the present invention. Such heart rhythm disturbances include supraventricular tachyarrhythmias, such as paroxysmal supraventricular tachycardia (PSVT) and atrial fibrillation/flutter (AF).

The controlled release formulation according to the invention preferably contains N⁶-cyclopentyl-5′-(N-ethyl)carboxamidoadenosine or a pharmaceutically acceptable salt or ester thereof in an amount effective to treat or prevent heart rhythm disturbance in a mammal. The oral dosage forms according to the invention are suitable for twice-daily or, preferably, once-daily administration of selodenoson. For example, once-daily oral formulation according to the invention may contain from about 0.1 mg to about 50 mg of N⁶-cyclopentyl-5′-(N-ethyl)carboxamidoadenosine or a pharmaceutically acceptable salt or ester thereof (calculated as mg of the active compound N⁶-cyclopentyl-5′-(N-ethyl)carboxamidoadenosine per dosage unit), preferably from about 1 mg to about 20 mg of N⁶-cyclopentyl-5′-(N-ethyl)carboxamidoadenosine or a pharmaceutically acceptable salt or ester thereof, and most preferably from about 3 mg to about 10 mg of N⁶-cyclopentyl-5′-(N-ethyl)carboxamidoadenosine or a pharmaceutically acceptable salt or ester thereof per dosage unit to provide a therapeutic amount of selodenoson throughout the day in a controlled release fashion. Advantageously, the once-daily oral formulation contains selodenoson from about 1 mg to about 3 mg per dosage unit, especially about 1 mg or about 3 mg per dosage unit.

The oral controlled release formulation according to the invention may be presented, for example, as tablets, multiparticulates, such as granules, spheroids, pellets, mini-tablets or sachets, capsules, or in any other suitable dosage form incorporating such multiparticulates. If desired, capsules such as hard or soft gelatin capsules, can contain the multiparticulates. If desired, the multiparticulate oral dosage forms can comprise a blend of at least two populations of spheroids, pellets or mini-tablets having different controlled-release in vitro selodenoson release profiles. If desired, one of the spheroid, pellet or mini-tablet populations can comprise immediate release selodenoson multiparticulates, such as multiparticulates formed by conventional means. A second population of spheroids, pellets or mini-tablets comprise controlled release selodenoson multiparticulates. Also, a population of spheroids, pellets or mini-tablets can comprise controlled release selodenoson multiparticulates coated with a controlled release or an enteric film coat. Further, the multiparticulates of the invention can be compressed into tablets.

The active ingredient in the formulation according to the invention is incorporated in a controlled release matrix. This may be any matrix that affords controlled release of selodenoson over at least a twelve hour period in vivo and preferably that affords in vitro dissolution rates of selodenoson within the ranges specified above.

Suitable materials for inclusion in a controlled release matrix include hydrophilic or hydrophobic polymers, such as gums, cellulose ethers, acrylic resins and protein derived materials. Examples of hydrophilic polymers to be used in this invention include hydroxyalkylcellulose, such as hydroxypropylcellulose and hydroxypropylmethylcellulose; poly(ethylene)oxide; alkylcellulose such as ethycellulose and methylcellulose; carboxymethylcellulose; hydrophilic cellulose derivatives; polyethylene glycol; polyvinylpyrrolidone; cellulose acetate; cellulose acetate butyrate; cellulose acetate phthalate; cellulose acetate trimellitate; polyvinylacetate phthalate; hydroxypropylmethylcellulose phthalate; and hydroxypropylmethylcellulose acetate succinate. Of these polymers, the cellulose ethers, especially hydroxyalkylcellulose, are preferred. Examples of hydrophobic polymers to be used in this invention include poly(alkyl methacrylate) and poly (vinyl acetate). Other suitable hydrophobic polymers include polymers or copolymers derived from acrylic or methacrylic acid esters, copolymers of acrylic and methacrylic acid esters, zein, and shellac. The formulation may conveniently contain about 1-90% by weight of one or more hydrophilic or hydrophobic polymers.

Also, suitable materials for controlled release matrix include digestible or non-digestible, saturated or unsaturated, long chain (C₈-C₅₀, especially C₁₂-C₄₀), substituted or unsubstituted hydrocarbons. Such digestible long-chain hydrocarbons include fatty acids, fatty alcohols, glyceryl esters of fatty acids, mineral oils, hydrogenated vegetable oils and waxes, hydrocarbons having a melting point of between 25 and 90° C. are preferred. Of these long chain hydrocarbon materials, hydrogenated vegetable oils and glyceryl esters of fatty acids are preferred. The formulation may conveniently contain up to 90% by weight of at least one digestible, long chain hydrocarbon, such as glyceryl behenate, hydrogenated cottonseed oil, castor oil, or hydrogenated castor oil.

Suitable hydroxypropylmethylcelluloses for use according to the invention preferably have a viscosity (2% w/v in water at 20° C.) of about 100 to 100,000 cps, preferably 100 to 30,000 cps. Especially suitable are Methocel® K and E types or their equivalents, preferably Methocel® K100M CR, Methocel® E4M, and Methocel® K4M CR. Suitable hydroxypropylcelluloses have a molecular weight of about 80,000 to 1,150,000, more preferably 80,000 to 600,000, such as Klucel® LF, which has a molecular weight of 100,000. Suitable poly(ethylene oxide) has a molecular weight of about 100,000 to 7,000,000, more preferably 900,000 to 7,000,000. Suitable ethylcelluloses have a viscosity of about 3 to 110 cps, more preferably 7 to 100 cps.

One particularly suitable controlled release matrix comprises one or more hydroxyalkylcelluloses. The hydroxyalkylcelluloses are preferably hydroxy(C₁ -C₄)alkyl (C₁-C₆)alkylcellulose, especially hydroxypropyl methylcellulose. The controlled release formulation according to the invention preferably contains from about 30% to about 75% by weight, especially from about 40% to about 70% by weight of dosage unit of one or more hydroxyalkylcellulose (calculated as the total amount of hydroxyalkylcelluloses by weight of dosage unit).

One particularly suitable controlled release matrix comprises two or more digestible, long chain hydrocarbons. The digestible, long chain hydrocarbons are preferably vegetable oils, such as hydrogenated vegetable oil, e.g., hydrogenated cotton seed oil (Lubritab®), and glyceryl esters of fatty acids, especially glyceryl behenate (Compritol® or Compritol®-888) and castor oil. The controlled release formulation according to the invention preferably contains from about 10% to about 65% by weight, especially from about 10% to about 50% by weight of dosage unit of one or more hydrogenated vegetable oil (calculated as the total amount of hydrogenated vegetable oils by weight of dosage unit), and from about 15% to about 50% by weight, especially from about 15% to about 45% by weight of dosage unit of one or more glyceryl ester of a fatty acid (calculated as the total amount of glyceryl esters of fatty acids by weight of dosage unit). Advantageously, the one or more glyceryl esters of fatty acid include castor oil in an amount of about 5-25% by weight of dosage unit, preferably about 10-20% by weight of dosage unit. Advantageously, the controlled release formulation including castor oil is in the form of multiparticulates.

The controlled release matrix may also contain other pharmaceutically acceptable ingredients, i.e., excipients, which are conventional in the pharmaceutical art such as diluents, lubricants, binders, granulating aids, colourants, flavourants, surfactants, pH adjusters, anti-adherents and glidants, e.g. dibutyl sebacate, ammonium hydroxide, oleic acid and colloidal silica. Suitable diluents include pharmaceutically acceptable inert fillers such as microcrystalline cellulose, lactose, dibasic calcium phosphate, saccharides, and/or mixtures of any of the foregoing. The diluent is suitably a water soluble diluent. Examples of diluents include microcrystalline cellulose such as Avicel® PH112, Avicel® PH101 and Avicel® PH102; lactose such as lactose monohydrate, lactose anhydrous, and Lactose Fast-Flo®; dibasic calcium phosphate; mannitol; starch; sorbitol; sucrose; and glucose. The diluent is preferably used in an amount of from 5 to 70% by weight of dosage unit, preferably from 10 to 45% by weight of dosage unit, of the controlled release formulation.

Suitable lubricants, including agents that act on the flowability of the powder to be compressed are, for example, colloidal silicon dioxide such as Aerosil 200, talc, stearic acid, magnesium stearate, and calcium stearate, castor oil and hydrogenated castor oil, and/or polyethylene glycol.

Suitable binders include starch paste, for example, maize starch, wheat starch, rice starch, potato starch, gelatin, tragacanth, methyl cellulose, hydroxypropylmethylcellulose, sodium carboxymethylcellulose, and/or polyvinyl pyrrolidone, polyethylene glycols such as PEG 6000, cetostearyl alcohol, cetyl alcohol, polyoxyethylene alkyl ethers, polyoxyethylene castor oil derivatives, polyoxyethylene sorbitan fatty acid esters, polyoxyethylene stearates, poloxamers, and waxes. If desired, disintegrating agents can be added, such as, the above-mentioned starches and also carboxymethyl-starch, cross-linked polyvinyl pyrrolidone, agar, or alginic acid or a salt thereof, such as, sodium alginate.

To improve the stability of N⁶-cyclopentyl-5′-(N-ethyl)carboxamidoadenosine in the controlled release formulation, an antioxidant compound can be included. Suitable antioxidants include sodium metabisulfite; tocopherols such as alpha-, beta-, or delta-tocopherol, tocopherol esters and alpha-tocopherol acetate; ascorbic acid or a pharmaceutically acceptable salt thereof; and ascorbyl palmitate.

The controlled release formulation according to the invention may optionally be film coated using any film coating material conventional in the pharmaceutical art. Preferably an aqueous film coating is used. The tablets, pellets, and spheroids may optionally be coated with concentrated saccharide solutions which may optionally contain gum arabic, talc, polyvinyl pyrrolidone, polyethylene glycol, and/or titanium dioxide, lacquer solutions and suitable organic solvents or solvent mixtures. If desired, the controlled release formulations of the invention, such as tablets or multiparticulates, can be coated with a controlled release polymer layer so as to provide additional controlled release properties. Suitable polymers that can be used to form this controlled release layer include the rate controlling polymers listed above. For example, in order to produce coatings resistant to gastric juices and to provide controlled release of the active agent, solutions of suitable water soluble or water insoluble cellulose preparations can be used. These cellulose preparations include ethyl cellulose, hydroxypropylmethylcellulose, hydroxypropylcellulose, acetylcellulose phthalate and hydroxypropylmethyl-cellulose phthalate. Also, suitable controlled release coating materials include water insoluble waxes and polymers such as polymethylacrylates (for example EUDRAGIT® polymers), or water soluble polymers such as polyvinylpyrrolidone. Optionally other water soluble agents such as polysorbate 80 may be added. The film coating may also contain excipients customary in film-coating procedures, such as light-protective pigments, for example iron oxide, or titanium dioxide, anti-adhesive agents, for example talc, and also suitable plasticizers such as PEG 400, PEG 6000, diethyl phthalate or triethyl citrate. Dye stuffs or pigments can also be added to the tablets or dragee coatings, for example, for identification or in order to characterize combinations of active compound doses.

Suitable EUDRAGIT® polymers for use as controlled release coating materials include Eudragit® L30, Eudragit® L100, and Eudragit® S100.

Useful amounts of such polymers in the controlled release tablet or multiparticulate are in the range of from about 5% to about 15% by weight.

Advantageously, the controlled release formulation according to the invention comprises an optionally film coated tablet or spheroids (pellets) containing the active ingredient, a controlled release matrix and a spheronizing agent.

The spheronizing agent may be any suitable pharmaceutically acceptable material or mixtures thereof which may be spheronized together with the active ingredient and the controlled release matrix to form spheroids (pellets). Suitable spheronizing agents include polyvinylpyrrolidone, i.e., povidone, such as Plasdone® K29/32, microcrystalline cellulose such as Avicel® PH112, Avicel® PH101 and Avicel® PH102, sodium carboxymethyl cellulose, sodium glycolate starch and dextrans.

One useful form of unit dose form in accordance with the invention comprises a capsule filled with controlled release spheroids (pellets) as described above. Suitable capsules include push-fit capsules made of gelatin, as well as soft, sealed capsules made of gelatin and a plasticizer, such as, glycerol or sorbitol. The push-fit capsules can contain a plurality of the controlled release spheroids (pellets) according to the invention that may optionally be mixed with fillers such as lactose, binders such as starches, and/or lubricants such as talc or magnesium stearate and, optionally, stabilizers.

The controlled release formulations according to the invention can be prepared by incorporating N⁶-cyclopentyl-5′-(N-ethyl)carboxamidoadenosine (selodenoson) or a pharmaceutically acceptable salt or ester thereof in a controlled release matrix. Accordingly, the controlled release formulation according to the invention can be manufactured by blending selodenoson or a pharmaceutically acceptable salt or ester thereof with the controlled release matrix including optional auxiliary excipients followed by direct compression.

Controlled release tablets according to the invention can be prepared, for example, by mixing or blending N⁶-cyclopentyl-5′-(N-ethyl)carboxamidoadenosine (selodenoson) or a pharmaceutically acceptable salt or ester thereof with the controlled release matrix and desired excipient(s), if any, using conventional procedures, e.g., using a Y-Cone or bin-blender, and compressing the resulting mixture according to conventional tabletting procedure using a suitable size tabletting mould into a plurality of tablets. Tablets can be produced using conventional tabletting machines, e.g., Manesty Betapress machine, standard single punch F3 Manesty machine or Kilian RLE15 rotary tablet machine. Advantageously, the active agent is screened through a suitable screen before mixing. Also, preferably, the controlled release materials including the excipients, such as the binder, are screened through a suitable mesh screen before mixing.

For example, a controlled release tablet according to the invention can be prepared by a direct compression method or by a wet granulation method. In the direct compression, the lubricant can be optionally mixed first with only a portion of the tablet blend and then mixing this blend with the remaining portion of the tablet blend before compressing into tablets (see Examples 6 and 7). In the wet granulation method, for example, selodenoson or a pharmaceutically acceptable salt or ester thereof is first mixed with one or more excipients and then with a binder-water solution to form granules. The wet granules are dried, milled, optionally sieved, and mixed with suitable excipients before compressing into tablets (see Example 7).

A controlled release formulation according to the invention may also be prepared in the form of multiparticulates, such as spheroids (pellets). The multiparticulates according to the invention can be prepared by granulating a mixture comprising N⁶-cyclopentyl-5′-(N-ethyl)carboxamidoadenosine or a pharmaceutically acceptable salt or ester thereof and one or more materials suitable for controlled release matrix, and shaping and compressing the granules. The resulting granules may be sieved to eliminate any over- or undersized particles.

Advantageously, multiparticulates according to the invention can be prepared by, for example,
(a) granulating a mixture comprising N⁶-cyclopentyl-5′-(N-ethyl)carboxamidoadenosine or a pharmaceutically acceptable salt or ester thereof, one or more digestible, long-chain hydrocarbons, one or more spheronizing agents, and optionally one or more excipients to give a granulated mixture;
(b) extruding the granulated mixture to give an extrudate;
(c) spheronizing the extrudate until a plurality of spheroids are formed;
(d) drying the plurality of spheroids; and
(e) optionally coating the spheroids with a film coat.

Advantageously, the one or more digestible, long-chain hydrocarbons in the above process include castor oil. Preferably, the amount of castor oil is about 5-25% by weight of dosage unit, more preferably about 10-20% by weight of dosage unit. Advantageously, the total amount of one or more digestible, long-chain hydrocarbons is from about 60% to about 90% by weight of dosage unit, preferably from about 70% to about 90% by weight of dosage unit. Advantageously, the film coating material is a poymethylacrylate, preferably an EUDRAGIT® polymer selected from the group consisting of Eudragit® L30, Eudragit® L100, and Eudragit® S100. Advantageously, the amount of the film coating material is from about 5% to about 15% by weight, preferably from about 8% to about 15% by weight, of the plurality of spheroids.

One advantageous form of unit dose formulation in accordance with the present invention comprises a capsule filled with controlled release particles comprising the active agent, a hydrophobic fusible carrier or diluent and a hydrophobic controlled release matrix. In particular, the controlled release particles are preferably prepared by a process using hot melt technology. The process comprises forming a mixture of about 20-80% by weight of the total amount of the active agent and melted fusible release control materials followed by cooling and milling the mixture to form a mixture A; forming a mixture of the rest of the active agent with a controlled release matrix and optional excipients to form a mixture B; mixing the mixture A with the mixture B and optionally one or more excipients, such as a lubricant and a spheronizing agent, to form a mixture C; granulating the mixture C to provide a granulated mixture; extruding the granulated mixture to give an extrudate; spheronizing the extrudate until a plurality of spheroids are formed; drying the plurality of spheroids; and optionally coating the plurality of spheroids with a film coat.

The hydrophobic fusible carrier or diluent should be a hydrophobic material such as a natural or synthetic wax or oil, for example hydrogenated vegetable oil, hydrogenated castor oil, Beeswax, Carnauba wax or glyceryl monostearate, and suitably has a melting point of from 35 to 140° C., preferably from 45 to 110° C. Advantageously, the hydrophobic fusible carrier or diluent is hydrogenated castor oil.

Another process for the manufacture of a formulation in accordance with the invention comprises
(a) mixing about 50% by weight of the total amount of N⁶-cyclopentyl-5′-(N-ethyl)carboxamidoadenosine (selodenoson) or a pharmaceutically acceptable salt or ester thereof and a melted hydrophobic, fusible carrier or diluent to provide a mixture A;
(b) cooling and milling the mixture A;
(c) mixing the remaining portion of the total amount of N⁶-cyclopentyl-5′-(N-ethyl)carboxamidoadenosine or a pharmaceutically acceptable salt or ester thereof, one or more digestible, long-chain hydrocarbons, and optionally one or more excipients to provide a mixture B;
(d) mixing the mixture A with the mixture B, one or more spheronizing agents and optionally one or more digestible, long-chain hydrocarbons to provide a mixture C;
(e) granulating the mixture C;
(f) extruding the granulated mixture C to give an extrudate;
(g) spheronizing the extrudate until a plurality of spheroids are formed;
(h) drying the plurality of spheroids; and
(i) optionally coating the plurality of spheroids with a film coat.

The resulting particles may be sieved to eliminate any over-or undersized material then formed into the desired dosage units by for example, encapsulation into hard gelatin capsules containing the required dose of the active agent.

Advantageously, the amount of fusible release control material added in step (a) is between about 3% and about 18% by weight of dosage unit, i.e., of the total amount of ingredients added in the entire manufacturing operation, more preferably between about 6% and about 15% by weight. In step (c), the amount of one or more digestible, long chain hydrocarbons is conveniently from about 30% to about 70% by weight of dosage unit, i.e., of the total amount of ingredients added in the entire manufacturing operation, preferably from about 40% to about 60% by weight of dosage unit. Advantageously, the one or more digestible, long chain hydrocarbons in step (c) are hydrogenated cotton seed oil and glyceryl behenate. Preferably, in step (d), at least one digestible, long chain hydrocarbon is mixed with the mixtures A and B, preferably castor oil. Preferably, the amount of castor oil is about 5-25% by weight of dosage unit, more preferably about 10-20% by weight of dosage unit. Advantageously, the total amount of digestible, long-chain hydrocarbons in the controlled release formulation prepared as described above is from about 35% to about 95% by weight of dosage unit, preferably from about 50% to about 85% by weight of dosage unit.

Step (a) of the process may be carried out by manual mixing, in low shear planetary mixers, or in conventional high speed mixers with a standard stainless steel interior, e.g. a Collette Vactron 75 or equivalent mixer.

If desired, the controlled release multiparticulates described above can be compressed into tablets using methods and excipients described above.

The release profile can be adjusted in a number of ways. For instance, by controlling the relative amounts of the components in the controlled release matrix it is possible to adjust the release profile of N⁶-substituted-5′-(N-substituted)carboxamidoadenosine (selodenoson) or a pharmaceutically acceptable salt or ester thereof. Also, the amount and the nature of the controlled release matrix, the diluent and the binder have an effect on the release profile. In particular, hydrophobic polymers have a stronger effect on slowing the release of the active agent than hydrophilic polymers. Further, the release rates of the active agent are faster from controlled release formulations comprising soluble diluents, such as lactose, compared to those comprising cellulose diluents, such as microcrystalline cellulose. The mean particle size and the distribution of the pellets containing the active agent is affected by the binder: more fine particles are obtained without using a binder. The release profile can also be adjusted by using micronized selodenoson.

Selodenoson can be synthesized according to the methods described in U.S. Pat. Nos. 5,310,731 and 4,868,160. For example, selodenoson may be obtained from 2′,3′-O-isopropylideneinosine-5′-carboxylic acid by treatment with a suitable inorganic acid halide, such as thionyl chloride, to yield the intermediate, 6-chloro-2′,3′-O-isopropylidene-9-β-D-ribofuranosylpurine-5′-carbonyl chloride. The intermediate, 6-chloro-2′,3′-O-isopropylidene-9-β-D-ribofuranosylpurine-5′-carbonyl chloride, (or the corresponding bromide, if, for example thionyl bromide is used instead of thionyl chloride) need not be isolated in a pure state.

The acid chloride moiety of 6-chloro-2′,3′-O-isopropylidene-9-β-D-ribofuranosylpurine-5′-carbonyl chloride (or the acid bromide of the corresponding bromo-analog) is significantly more readily displaced by nucleophilic reagents than the halide in the 6-position of the purine moiety. Therefore, 6-chloro-2′,3′-O-isopropylidene-9-β-D-ribofuranosylpurine-5′-carbonyl chloride is first reacted with ethylamine to yield an intermediate as a substituted carboxamide, wherein the halide is retained in the 6-position of the purine moiety.

The intermediate substituted carboxamide is subsequently reacted with cyclopentylamine, and the isopropylidene blocking group is removed with dilute acid to yield selodenoson having free hydroxyl groups in the 2′ and 3′ positions of the ribofuranose moiety. Instead of the isopropylidene blocking group, other acid-stable blocking groups can also be used to protect the 2′-hydroxyl and 3′-hydroxyl groups of the ribofuranose moiety during the step of treatment with the inorganic acid halide.

The present invention is also directed to the use of N⁶-cyclopentyl-5′-(N-ethyl)carboxamidoadenosine or a pharmaceutically acceptable salt or ester thereof in the manufacture of an oral controlled release formulation for treating heart rhythm disturbances in a mammal.

The controlled release formulations can, if desired, also contain other compatible therapeutic agents. Examples of useful therapeutic agents that can be co-administered with active compounds of the present invention include verapamil, quinidine, procainamide, diisopyramide, flecanide, ibutilide, dofetilide, amiodarone, sotalol, diltiazem, esmolol, propranolol, metoprolol, and digoxin.

### EXAMPLES

The following examples are illustrative, but not limiting, of the method and compositions of the present invention. Suitable modifications and adaptations of the variety of conditions and parameters normally encountered and obvious to those skilled in the art are within the spirit and scope of the invention.

Procedure for measuring in vitro dissolution rates for controlled release tablets

The in vitro release rates for tablets were measured using Apparatus 3, i.e., reciprocating cylinder as described in the Unites States Pharmacopeia (USP) 23 (p. 1793-1794, United States Pharmacopeial Convention, Inc. (1995)). Accordingly, selodenoson containing controlled release tablets were placed in the dissolution medium with the dissolution conditions as follows:
Temperature: 37.0±0.5° C.;
Speed: 30 dips/minute;
Start dip wait (first vessel): 10 seconds;
Drain time (between vessels): 1 minute;
Vessel size: 300 mL;
Medium volume: 250 mL;
Vankel Bio-Dis Dissolution Test
Station Reciprocating Cylinder: 300 mL vessels fitted with a stainless steel screen; and

Dissolution media and sampling timepoints were as described in Table

TABLE 6

| Timepoint | | Dissolution Medium |
|---|---|---|
| 15 | minutes | 0.1 M HCl |
| 30 | minutes | 0.1 M HCl |
| 45 | minutes | 0.1 M HCl |
| 60 | minutes | 0.1 M HCl |
| 2 | hours | 0.1 M HCl |
| 4 | hours | pH 6.8 phosphate buffer |
| 8 | hours | pH 6.8 phosphate buffer |
| 12 | hours | pH 6.8 phosphate buffer |

Accordingly, 30 dissolution vessels were filled with 250 mL of the first dissolution medium, 0.1 M HCl, using a graduated cylinder. The level of the medium in each vessel was marked with a non-soluble marker. All the 30 vessels were loaded into rows 1-5 of the dissolution apparatus bath (37.0±0.5° C). 18 dissolution vessels were filled with the second medium, pH 6.8 phosphate buffer (KH₂PO₄), and put on the side. The level of the medium in each vessel was marked with a non-soluble marker. When the first medium reached 37.0±0.5° C., 6 tablets were weight and placed into separate reciprocating cylinders and attached to the Bio-Dis plungers corresponding to columns 1-6 of the dissolution apparatus bath. The first program was started for up to 30 minutes profile time. The second program was started immediately at the end of the first program.

At the end of the first program (at 30 minutes), the head of the Bio-Dis was raised and the reciprocating cylinders were held above row 2 of the dissolution vessels. The reciprocating cylinders were allowed to drain for 1 minute before proceeding to the next step. After the draining, the first two rows of dissolution vessels were removed and replaced with the vessels from rows 4 and 5 (containing 0.1 M HCl). The removed vessels were put aside.

The 18 vessels containing the second dissolution medium, pH 6.8 phosphate buffer, into rows 4-6 of the Bio-Dis. The second program was started for the profile 30 minutes to 12 hours. This program began at row 1 of dissolution vessels. At the end of the second program, all the vessels were removed from the Bio-Dis and allowed to cool to room temperature.

The appropriate dissolution medium (i.e., 0.1 M HCl to those vessels containing 0.1 M HCl and pH 6.8 phosphate buffer to those vessels containing pH 6.8 phosphate buffer) to each vessel to bring the level of medium back to the mark made in the beginning. This step corrects for the evaporation of medium before analysis. Each vessels was thoroughly mixed with a glass stir rod. An aliquot was taken from each vessel and filtered through a 0.45 μm PVDF syringe filter, discarding the initial 1-2 mL, and the remaining filtrate was collected into an HPLC vial for analysis.

Procedure for measuring in vitro dissolution rates for capsules filled with controlled release multiparticulates

The in vitro release rates for capsules filled with controlled release multiparticulates were measured using Apparatus 2, i.e., the paddle method, as described in the Unites States Pharmacopeia (USP) 23 (p. 1791-1793, United States Pharmacopeial Convention, Inc. (1995)). The dissolution conditions were as follows:
Temperature: 37.0±0.5° C.;
Speed: 50 rpm;
Volume: 900 mL; and
Dissolution media and sampling timepoints were as described above in Table 6.

The dissolution test for each sample was conducted in a vessel containing 500 mL of 0.1 M HCl and dissolution data was collected for 0-120 minutes. After 2 hours, the 0.1 M HCl was neutralized 10 M NaOH solution and pH 6.8 buffer was then added to a total volume of 900 mL. The dissolution testing was continued with the pH 6.8 phosphate buffer.

In particular, the procedure for measuring the in vitro release rates for capsules containing 3 mg of selodenoson was as follows. Each tested capsule was opened and the capsule shell and the contents were dropped into a dissolution vessel containing 500 mL of pre-warmed (37.0±0.5° C.) 0.1 M HCl. The rotation was started immediately. 5 mL of the solution was withdrawn from each vessel through a 10 μm Full Flow™ Filter at 15, 30, 45, 60, and 120 minutes, respectively. The 5 mL of pre-warmed dissolution medium was added to each vessel to replace the withdrawn amount after every pull point. After the 120 minute (2 hour) sample was withdrawn and the 5 mL 0.1 M HCl was replaced, 5 mL of 10 M NaOH solution was added into each vessel and stirred for 2 minutes to neutralize the 0.1 M HCl. The paddle rotation was stopped and the head of the dissolution apparatus was raised. 395 mL of phosphate buffer (38.8 g of KH₂PO₄ in 2 L of water mixed with 13.0 mL of 10 M NaOH and 487 mL of water added) pre-warmed at 37.0±0.5° C. was added to each vessel. The final composition of the medium was equivalent of pH 6.8 phosphate buffer. The head of the dissolution apparatus was lowered and the rotation was again begun. 5 mL of the solution was withdrawn from each vessel as described above at 4, 8, and 12 hours. 5 mL of pre-warmed pH 6.8 phosphate buffer was added to each vessel the replace the withdrawn amount after every pull point. Each collected sample was filtered through a 0.45 μm PVDF syringe filter, discarding the first 1 to 2 mL, and the filtrate was collected into an HPLC vial for analysis.

The procedure for measuring the in vitro release rates for capsules containing 1 mg of selodenoson was as follows. The dissolution media and sampling timepoints were as described in Table 7:

TABLE 7

| Timepoint | | Dissolution Medium |
|---|---|---|
| 15 | minutes | 0.005% SDS - 0.1 M HCl |
| 30 | minutes | 0.005% SDS - 0.1 M HCl |
| 45 | minutes | 0.005% SDS - 0.1 M HCl |
| 60 | minutes | 0.005% SDS - 0.1 M HCl |
| 2 | hours | 0.005% SDS - 0.1 M HCl |
| 4 | hours | pH 6.8 phosphate buffer |
| | | containing 0.5% SDS |
| 8 | hours | pH 6.8 phosphate buffer |
| | | containing 0.5% SDS |
| 12 | hours | pH 6.8 phosphate buffer |
| | | containing 0.5% SDS |

A Vankel VK7000 Dissolution Test Station was used, but any equivalent test station can be used. A sinker having an 12.0×0.2 mm inside diameter and a 25-26 mm length was used. The trunk of the sinker consisted of a spiral 3.0-3.5 mm pitch coil made with an acid-resistant 1 mm thick wire. The spiral was supported on the outside with 10 wires that were fixed almost in parallel and with equal spacing. The sides of the sinker were fixed with two double wires in a cross shape, but on one of its sides it was fixed with a clasp that could be opened so that a sample could be inserted.

Each tested capsule was placed in a sinker and dropped into a dissolution vessel containing 500 mL of pre-warmed (37.0±0.5° C.) 0.005% SDS-0.1 M HCl (10 mL of 2.0% SDS (sodium dodecyl sulfate) mixed and 34 mL of conc. HCl diluted in 4 L of water). The rotation was started immediately. 5 mL of the solution was withdrawn from each vessel through a 10 μm Full Flow™ Filter at 15, 30, 45, 60, and 120 minutes, respectively. 5 mL of pre-warmed dissolution medium was added to each vessel to replace the withdrawn amount after every pull point. After the 120 minute (2 hour) sample was withdrawn and the 5 mL 0.005% SDS-0.1 M HCl was replaced, 5 mL of 10 M NaOH solution was added into each vessel and stirred for 2 minutes to neutralize the 0.1 M HCl. The paddle rotation was stopped and the head of the dissolution apparatus was raised. 395 mL of 1.14% SDS (sodium dodecyl sulfate (OmniPur, EM Science))-phosphate buffer (38.8 g of KH₂PO₄ in 2 L of water mixed with 13.0 mL of 10 M NaOH and 487 mL of water added) pre-warmed at 37.0±0.5° C. was added to each vessel. The final composition of the medium was equivalent of pH 6.8 phosphate buffer containing 0.5% SDS. The head of the dissolution apparatus was lowered and the rotation was again begun. 5 mL of the solution was withdrawn from each vessel as described above at 4, 8, and 12 hours (2, 6, and 10 hours after medium change). 5 mL of pre-warmed phosphate buffer was added to each vessel the replace the withdrawn amount after every pull point. Each collected sample was filtered through a 0.45 μm PVDF syringe filter, discarding the first 1 to 2 mL, and the filtrate was collected into an HPLC vial for analysis.

HPLC Analysis

Samples of the dissolution media were taken at the timepoints specified above and analyzed by high performance liquid chromatography (HPLC) to quantify the amount of selodenoson cumulatively released. The HPLC parameters were as follows:
Column: Waters Symmetry Shield RP-8, 5 μm, 150 mm×3.9 mm or equivalent;
Detection: UV at 272 nm;
Column temperature: 40° C.;
Injection volume: 20 μL (3 mg selodenoson formulations) or 50 μL (1 mg selodenoson formulations);
Flow rate: 1.0 mL/min;
Run time: 20 min; and
Mobile phase: 5 mM tetrabutylammonium phosphate (TBA, Aldrich) (pH 6.5): CH₃CN=80:20.

When analyzing the 1 mg selodenoson multiparticulate formulations the following injector program was used:
needle wash in vial 100 (1.14% SDS-phosphate buffer) 2 times;
draw 50.0 μL from sample;
draw 40.0 μL from vial 99 (1.14% SDS-phosphate buffer);
mix 90.0 μL in seat, 2 times; and
overlap injection cycle 16.0 minutes after injection.

### Example 1

### Hydropholic Tablets

Hydrophilic controlled release tablets were prepared using components described in Table 8 having 3 mg N⁶-cyclopentyl-5′-(N-ethyl)carboxamidoadenosine per tablet.

TABLE 8
The ingredients of hydrophilic controlled release
tablets containing 3 mg of selodenoson.

| | Tablet 1 | Tablet 2 |
|---|---|---|
| Ingredient | mg/tablet | mg/tablet |
| Selodenoson | 3.00 | 3.00 |
| Methocel ® K100M CR | — | 140.00 |
| Methocel ® E4M | 48.00 | 40.00 |
| Methocel ® K4M CR | 200.00 | — |
| Lactose Fast-Flo ® | 100.00 | 140.00 |
| Microcrystalline Cellulose | 43.00 | 71.00 |
| (Avicel ® PH102) | | |
| Colloidal Silicon Dioxide | 2.00 | 2.00 |
| Magnesium Stearate | 4.00 | 4.00 |
| Total Tablet Weight/mg | 400.00 | 400.00 |

Selodenoson was pre-screened through a #60 mesh screen and mixed geometrically with Lactose Fast-Flo® (Merial Ltd., N.J., USA). Methocel® K100M CR (Dow Chemical, Midland, Mich., USA), Methocel® K4M CR, and Avicel® PH102 (FMC Corporation, Philadelphia, Pa., USA) were screened through a #40 mesh screen, added to the selodenoson mixture and mixed for 5 minutes. Colloidal silicon dioxide and magnesium stearate were added and mixed for 2 minutes to yield the final blend. The final blend was compressed into tablets using Manesty Betapress (Manesty, UK) equipped with 0.565 inch×0.2671 inch modified capsule shape tooling to achieve the desired tablet weight.

### Example 2

### Hydrophobic Tablets

Hydrophobic controlled release tablets were prepared using components described in Table 9 having 3 mg selodenoson per tablet.

TABLE 9
The ingredients of hydrophobic controlled release
tablets containing 3 mg of selodenoson.

| | Tablet 3 | Tablet 4 |
|---|---|---|
| Ingredient | mg/tablet | mg/tablet |
| Selodenoson | 3.00 | 3.00 |
| Glyceryl Behenate | 150.00 | 120.00 |
| Lubritab ® | 80.00 | 150.00 |
| Povidone K29/32 | 9.00 | 9.00 |
| Lactose Fast-Flo ® | 80.00 | 80.00 |
| Microcrystalline Cellulose | 70.00 | 30.00 |
| (Avicel ® PH102) | | |
| Colloidal Silicon Dioxide | 4.00 | 4.00 |
| Magnesium Stearate | 4.00 | 4.00 |
| Total Tablet Weight/mg | 400.00 | 400.00 |

Selodenoson was pre-screened through a #60 mesh screen and mixed geometrically with microcrystalline cellulose. Glyceryl behenate, Lubritab® (Penwest, N.Y., USA), Povidone K29/32 (ISP Technologies, Texas, USA), and Lactose Fast-Flo® were screened through a #40 mesh screen, added to the selodenoson mixture and mixed for 5 minutes. Colloidal silicon dioxide and magnesium stearate were added and mixed for 2 minutes to yield the final blend. The final blend was compressed into tablets as described in Example 1.

### Example 3

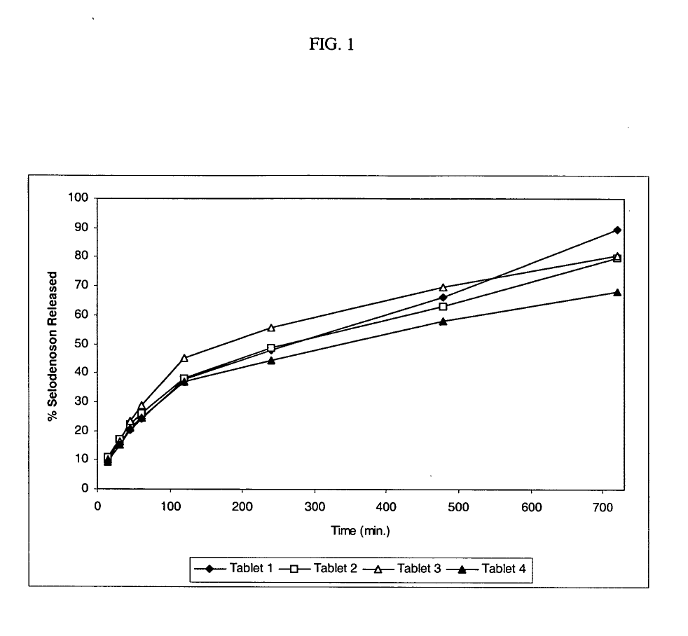

In vitro dissolution studies were conducted as described above on the tablets described in Examples 1 and 2. The results are shown in Table 10 below. The results are also shown graphically in FIG. 1.

TABLE 10
Dissolution of the controlled release tablets containing
selodenoson prepared in Examples 1 and 2.

Mean wt- % of selodenoson released

| Time/ | Tablet 1 | Tablet 2 | Tablet 3 | Tablet 4 |
|---|---|---|---|---|
| Minutes | (n = 6) | (n = 6) | (n = 3) | (n = 3) |
| 15 | 9.5 | 10.9 | 10.1 | 9.4 |
| 30 | 15.0 | 17.0 | 16.2 | 15.0 |
| 45 | 20.1 | 22.0 | 23.5 | 21.0 |
| 60 | 24.2 | 25.9 | 28.9 | 24.7 |
| 120 | 37.7 | 38.0 | 45.3 | 36.9 |
| 240 | 47.8 | 48.5 | 55.7 | 44.3 |
| 480 | 66.1 | 63.0 | 69.8 | 57.9 |
| 720 | 89.7 | 80.0 | 80.4 | 68.2 |

### Example 4

### Capsules

Controlled release capsules were prepared using components described in Table 11 having 3 mg selodenoson per capsule.

TABLE 11
The ingredients of controlled release capsules
containing 3 mg of selodenoson.

| | Capsule 1 | Capsule 2 | Capsule 3 |
|---|---|---|---|
| Ingredient | mg/capsule | mg/capsule | mg/capsule |
| Selodenoson | 3.00 | 3.00 | 3.00 |
| Glyceryl Behenate | 150.00 | 100.00 | 100.00 |
| (Compritol ®) | | | |
| Lubritab ® | 200.00 | 170.00 | 170.00 |
| Lactose Fast-Flo ® | 68.00 | 33.00 | 33.00 |
| Microcrystalline Cellulose | — | 45.00 | 65.00 |
| (Avicel ® PH102) | | | |
| Povidone K29/32 | 9.00 | 9.00 | 9.00 |
| Castor Oil | 70.00 | 80.00 | 80.00 |
| Hydrogenated Castor Oil | — | 60.00 | 40.00 |
| Capsule Fill Weight/mg | 500.00 | 500.00 | 500.00 |

Capsule 1 was prepared by mixing manually selodenoson with glyceryl behenate, Lubritab® and lactose to yield a mixture. The mixture was mixed with castor oil and granulated with povidone/water solution (15% w/v). The wet mass was extruded and marumerized to yield spherical particles. The spheres were dried in a fluid-bed dryer at 60° C. for 30 minutes. The dried granules were filled in suitable size hard gelatin capsules to yield the final product.

Capsules 2 and 3 were prepared as follows. A half required quantity of selodenoson was dispersed in melted hydrogenated castor oil, cooled and milled using a Fitz mill. The other half quantity of selodenoson was mixed with glyceryl behenate, Lubritab®, Avicel® PH102 and lactose. These two mixtures were mixed together with castor oil and granulated with povidone/water solution to form a wet mass. The wet mass was extruded and marumerized to yield spherical particles. The spheres were dried in a fluid-bed dryer. The dried granules were filled in suitable sized hard gelatin capsules to yield the final product.

### Example 5

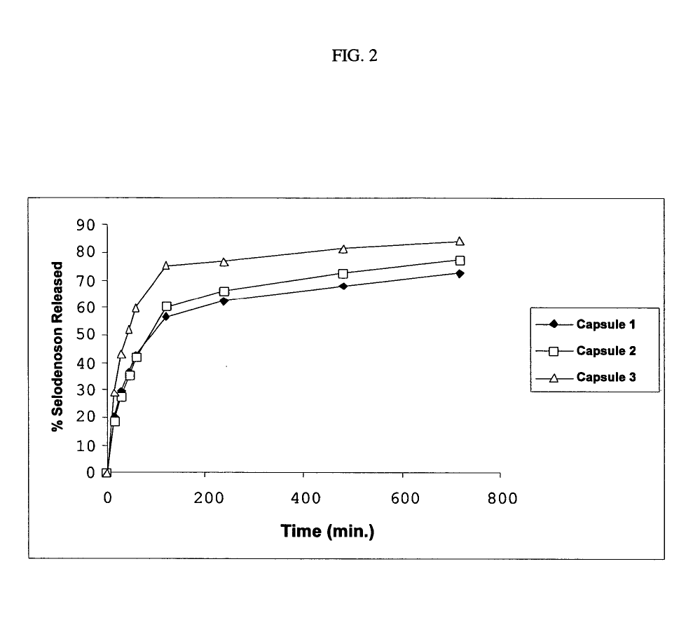

In vitro dissolution studies were conducted as described above on the capsules prepared in Example 4. The results are shown in Table 12 below. The results are also shown graphically in FIG. 2.

TABLE 12
Dissolution of the controlled release capsules
containing selodenoson prepared in Example 4.

| | Mean wt-% of selodenoson released | | |
|---|---|---|---|
| Time/ | Capsule 1 | Capsule 2 | Capsule 3 |
| minutes | (n = 6) | (n = 6) | (n = 6) |
| 15 | 20.3 | 18.7 | 29.1 |
| 30 | 29.5 | 27.6 | 43.1 |
| 45 | 36.5 | 35.3 | 51.9 |
| 60 | 42.6 | 41.9 | 59.9 |
| 120 | 56.9 | 60.5 | 75.7 |
| 240 | 62.4 | 66.3 | 77.1 |
| 480 | 68.2 | 72.8 | 81.5 |
| 720 | 72.5 | 77.2 | 84.4 |

### Example 6

### Hydrophilic Tablet Containing 1 mg of Selodenoson

A hydrophilic controlled release tablet was prepared using components described in Table 13 having 1 mg of N⁶-cyclopentyl-5′-(N-ethyl)carboxamidoadenosine.

TABLE 13
The ingredients of a hydrophilic controlled release
tablet 5 containing 1 mg of selodenoson.

Tablet 5

| Ingredient | mg/tablet | g/batch |
|---|---|---|
| Selodenoson | 1.00 | 1.00 |
| Methocel ® E4M | 200.00 | 200.00 |
| Methocel ® K4M CR | 50.00 | 50.00 |
| Lactose Fast-Flo ® | 100.00 | 100.00 |
| Microcrystalline Cellulose | 43.00 | 43.00 |
| (Avicel ® PH102) | | |
| Colloidal Silicon Dioxide | 2.00 | 2.00 |
| Magnesium Stearate | 4.00 | 4.00 |
| Total | 400.00 | 400.00 |

Tablet 5 was prepared by direct compression as follows. Selodenoson was screened through a #40 mesh screen and mixed geometrically with Lactose Fast-Flo® in a 1 qt. V-Blender. Lactose Fast-Flo® was added into the mixer in portions and the mixture was mixed for 2 minutes after each addition. The blender was finally rinsed with the last portion of Lactose Fast-Flo® and the whole blend was taken out in a polyethylene bag. Avicel® PH102 and colloidal silicon dioxide were mixed in a 1 qt. V-Blender for 5 minutes, sieved through a #20 mesh screen, and the mixture was kept in a separate polyethylene bag. The above prepared two mixtures were transferred in a 4 qt. V-Blender. Methocel® K4M CR and Methocel® E4M CR were sieved through a #20 mesh screen and added to the blender. The components were mixed for 20 minutes. Approximately half of the mixture was taken out in a polyethylene bag. Magnesium stearate was added to the remaining portion in the blender after sieving through a #30 mesh screen, and this mixture was mixed for 5 minutes. The separated portion was added back into the blender and the mixture was mixed for another 5 minutes to yield the final blend. The final blend was discharged in a polyethylene bag and compressed into tablets using Manesty Betapress (Manesty, UK) equipped with 0.565 inch×0.2671 inch plain modified capsule shaped tooling to achieve the desired tablet weight of 400 mg.

### Example 7

### Hydrophobic Tablets Containing 1 mg of Selodenoson

Hydrophobic controlled release tablets were prepared using the components described in Table 14 having 1 mg of N⁶-cyclopentyl-5′-(N-ethyl)carboxamidoadenosine.

TABLE 14
The ingredients of hydrophobic controlled release
tablets containing 1 mg of selodenoson.

| | Tablet 6 | Tablet 7 |
|---|---|---|
| Ingredient | mg/tablet | mg/tablet |
| Selodenoson | 1.00 | 1.00 |
| Compritol ®-888 | 150.00 | 150.00 |
| Lubritab ® | 80.00 | 80.00 |
| Microcrystalline Cellulose | 64.00 | 74.00 |
| (Avicel ® PH102) | | |
| Lactose Fast-Flo ® | 80.00 | 80.00 |
| Povidone K29/32 | 9.00 | 9.00 |
| Magnesium Stearate | 6.00 | 4.00 |
| Colloidal Silicon Dioxide | 10.00 | 2.00 |
| Total | 400.00 | 400.00 |

Tablet 6 was prepared by a direct compression method and tablet 7 was prepared by a wet granulation method as follows.

The Preparation of Tablet 6

Selodenoson was screened through a #40 mesh screen and mixed geometrically with Lactose Fast-Flo® in a 1 qt. V-Blender. Lactose Fast-Flo® was added into the mixer in portions and the mixture was mixed for 2 minutes after each addition. The blender was finally rinsed with the last portion of Lactose Fast-Flo® and the whole blend was taken out in a polyethylene bag. Avicel® PH102 and colloidal silicon dioxide were mixed in a 1 qt. V-Blender for 5 minutes, and sieved through a #20 mesh screen. The mixture was kept in a separate polyethylene bag. The mixtures from the above two steps were transferred in a 4 qt. V-Blender. Compritol®-888, Lubritab®, and Povidone K29/32 were added into the same blender after sieving through a #20 mesh screen. The components were mixed for 20 minutes. Approximately half of the mixture was taken in a polyethylene bag. Magnesium stearate was added to the remaining portion in the blender after sieving through a #30 mesh screen, and this mixture was mixed for 5 minutes. The separated portion was added back into the blender and mixing was continued for another 5 minutes to yield the final blend. The final blend was discharged in a polyethylene bag and compressed into tablets using Manesty Betapress (Manesty, UK) equipped with 0.565 inch×0.2671 inch plain modified capsule shaped tooling to achieve the desired tablet weight of 400 mg.

The Preparation of Tablet 7

Selodenoson was screened through a #40 mesh screen and mixed geometrically with Lactose Fast-Flo® in a polyethylene bag for 2 minutes. Avicel® PH102, Compritol®-888, and Lubritab® were sieved through a #20 mesh screen and mixed with the above mixture for 2 minutes. The mixture was transferred to a planetary mixer (Eureka Planetary Mixer) and mixed for 5 minutes. While mixing, povidone/water solution (15% w/v) was added to the mixer. The mixing was continued for 5 more minutes and additional water was added to the mixture until the granulation point was achieved. The granules were dried in a fluid-bed dryer at 60° C. for 45 minutes. The dried granules were milled (Fitz-Mill), and the milled granules were mixed with colloidal silicon dioxide and magnesium stearate for 2 minutes in a polyethylene bag to obtain the final blend. The final blend was compressed into tablets using Manesty Betapress (Manesty, UK) equipped with 0.565 inch×0.2671 inch plain modified capsule shaped tooling to achieve the desired tablet weight of 400 mg.

### Example 8

### Capsules Containing 1mg of Selodenoson

Controlled release capsules were prepared using the components described in Table 15 having 1 mg of selodenoson per capsule.

TABLE 15
The ingredients of controlled release capsules
containing 1 mg of selodenoson.

| | Capsule 4 | Capsule 5 |
|---|---|---|
| Ingredient | mg/capsule | mg/capsule |
| Selodenoson | 1.00 | 1.00 |
| Compritol ®-888 | 50.00 | 33.00 |
| Lubritab ® | 67.00 | 57.00 |
| Lactose Fast-Flo ® | 23.00 | 11.00 |
| Microcrystalline Cellulose | — | 15.00 |
| (Avicel ® PH102) | | |
| Povidone K29/32 | 3.00 | 3.00 |
| Castor Oil | 23.00 | 27.00 |
| Hydrogenated Castor Oil | — | 20.00 |
| Capsule Fill Weight/mg | 167.00 | 167.00 |

Capsule 4 was prepared by mixing selodenoson screened through a #40 mesh screen with lactose in a polyethylene bag for 2 minutes to yield a mixture. Compritol®-888 and Lubritab® were screened through a #20 mesh screen, added to the mixture, and the mixture was mixed for 5 minutes. The resulting mixture was transferred to a planetary mixer and mixed for 2 minutes while adding castor oil. The mixture was mixed for another 2 minutes and Povidone K29/32 solution in water (15% w/v) was added to the mixture. The wet mass was passed through an extruder to get extrudates. The extrudates were passed through a marumerizer to obtain pellets. The pellets were dried in a fluid-bed dryer (Strea S1) at 60° C. for 30 minutes. The dried pellets were screened through a #10 mesh screen and pellets retained on a #18 mesh screen were filled in size 2 hard gelatin capsules with a fill weight of 167 mg.

Capsule 5 were prepared as follows. 50% of the amount of selodenoson was dispersed in melted hydrogenated castor oil, allowed to cool while stirring, and milled using a Fitz mill to obtain the first mixture. The other 50% of selodenoson was geometrically mixed with lactose. Avicel® PH102, Compritol®-888, Lubritab®, and the first mixture were added, and mixing in a polyethylene bag was continued for 2 minutes. This mixture was transferred to a planetary mixer and mixed for 5 minutes while adding castor oil to the mixture. The mixture was granulated with povidone/water solution (15% w/v) while mixing was continued for another 5 minutes. The resulting mass was passed through an extruder to get extrudates. The extrudates were passed through a marumerizer to obtain pellets. The pellets were dried in a fluid-bed dryer (Strea S1) at 60° C. for 30 minutes. The dried pellets were screened through a #10 mesh screen and pellets retained on a #18 mesh screen were filled in size 2 hard gelatin capsules with a fill weight of 167 mg.

### Example 9

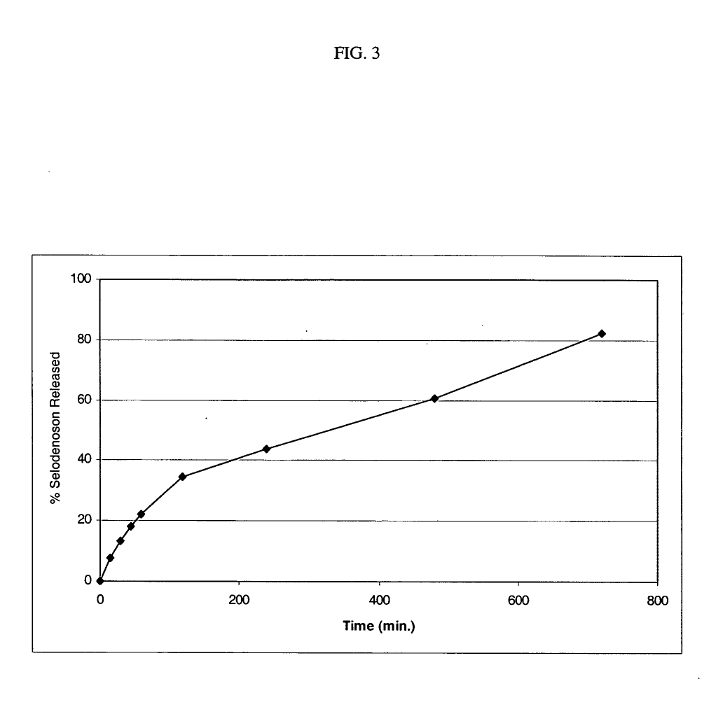

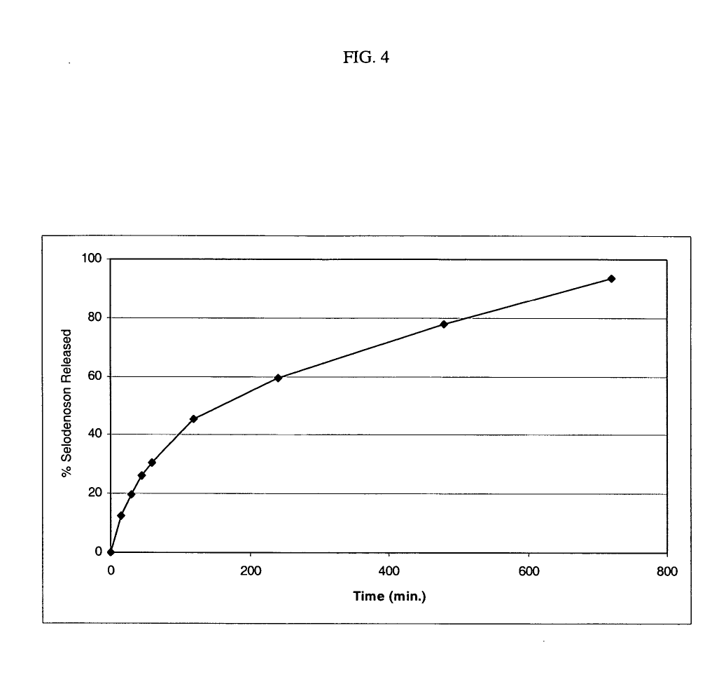

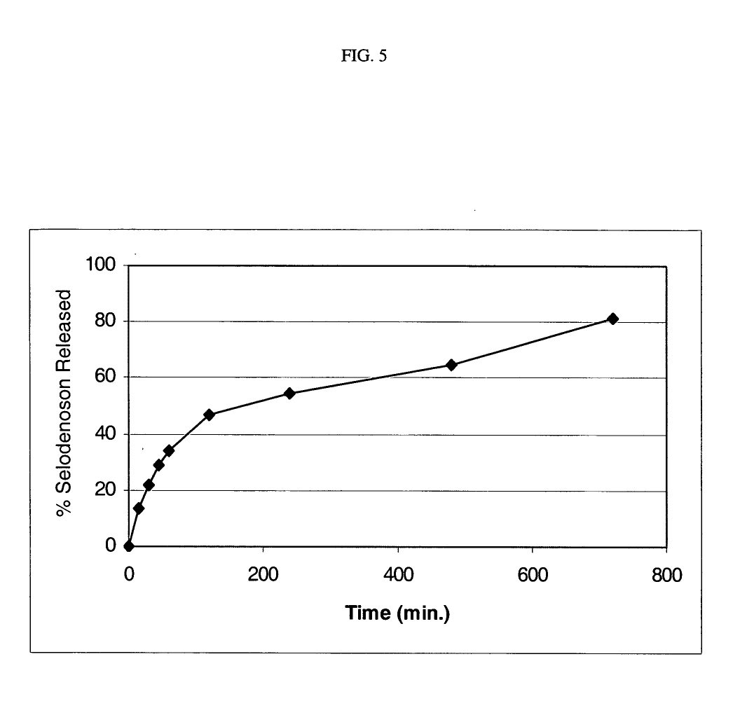

In vitro dissolution studies were conducted as described above on the tablets described in Examples 6 and 7. The results are shown in Table 16 below. The results are also shown graphically in FIGS. 3-5.

TABLE 16
Dissolution of the controlled release tablets containing
selodenoson prepared in Examples 6 and 7.

| | Mean wt-% of selodenoson released | | |
|---|---|---|---|
| Time/ | Tablet 5 | Tablet 6 | Tablet 7 |
| minutes | (n = 6) | (n = 6) | (n = 6) |
| 15 | 7.8 | 12.4 | 13.2 |
| 30 | 13.2 | 19.7 | 21.5 |
| 45 | 18.2 | 26.1 | 28.9 |
| 60 | 22.2 | 30.7 | 33.8 |
| 120 | 34.5 | 45.4 | 46.5 |
| 240 | 43.6 | 59.5 | 54.6 |
| 480 | 60.5 | 78.1 | 64.8 |
| 720 | 82.2 | 93.4 | 81.5 |

### Example 10

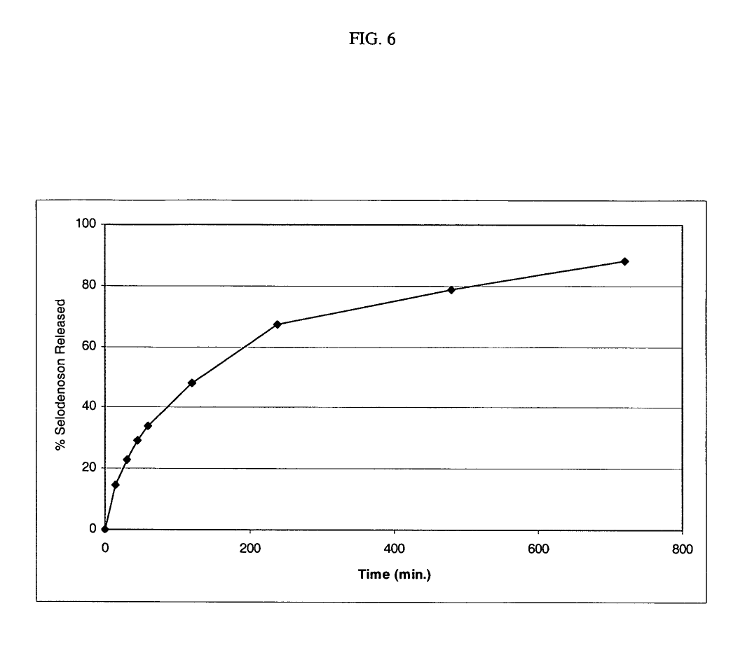

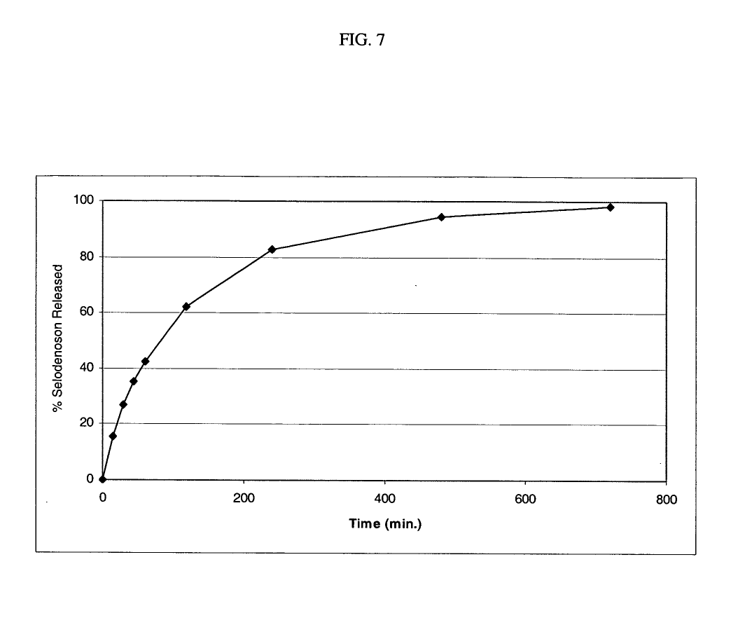

In vitro dissolution studies were conducted as described above on the capsules prepared in Example 8. The results are shown in Table 17 below. The results are also shown graphically in FIGS. 6-7.

TABLE 17
Dissolution of the controlled release capsules
containing selodenoson prepared in Example 8.

Mean wt-% of selodenoson released

| Time/ | Capsule 4 | Capsule 5 |
|---|---|---|
| Minutes | (n = 6) | (n = 6) |
| 15 | 14.6 | 15.5 |
| 30 | 22.8 | 27.0 |
| 45 | 29.1 | 35.3 |
| 60 | 33.9 | 42.6 |
| 120 | 48.2 | 62.0 |
| 240 | 67.2 | 82.8 |
| 480 | 78.9 | 94.4 |
| 720 | 88.1 | 98.3 |

### Example 11

### Capsules Containing Controlled Release Coated Pellets

Controlled release capsules containing selodenoson 1 mg per capsule were prepared using the components described in Table 15 for Capsule 4. Capsule 6 was prepared according to the procedure described for Capsule 4 in Example 8. The dried pellets were sprayed with Eudragit L100 so that the pellets contained Eudragit® L100 of 11% by weight of the pellets. Capsule 7 was prepared similarly and sprayed with Eudragit® L100 so that the pellets contained Eudragit® L100 of 15% by weight of the pellets. The sprayed pellets were screened through a #10 mesh screen and pellets retained on a #18 mesh screen were filled in size 2 hard gelatin capsules.

In vitro dissolution studies were conducted as described above on Capsule 6 and capsule 7. The results are shown in Table 18 below.

TABLE 18
Dissolution of the controlled release capsules
containing 1 mg of selodenoson prepared above.

Mean wt-% of selodenoson released

| Time/ | Capsule 6 | Capsule 7 |
|---|---|---|
| Minutes | (n = 6) | (n = 6) |
| 15 | 1 | 0.8 |
| 30 | 1.5 | 1.2 |
| 45 | 2 | 1.5 |
| 60 | 2.6 | 1.6 |
| 120 | 10.9 | 3.2 |
| 240 | 48.6 | 34.4 |
| 480 | 64.6 | 56 |
| 720 | 75.6 | 72 |

The controlled release formulations of the present invention thus provide an effective delivery system for the once daily administration of N⁶-cyclopentyl-5′-N-ethyl)carboxamidoadenosine (selodenoson) to patients in need of such treatment. The formulations of the invention are expected to provide substantial level plasma concentrations of N⁶-cyclopentyl-5′-(N-ethyl)carboxamidoadenosine (selodenoson) falling within the therapeutic range of the drug over a period which permits administration once daily.

Having now fully described this invention, it will be understood to those of ordinary skill in the art that the same can be performed within a wide and equivalent range of conditions, formulations, and other parameters without affecting the scope of the invention or an embodiment thereof. All patents, patent applications, and publications cited herein are fully incorporated by reference herein in their entirety.

1. A controlled release pharmaceutical formulation suitable for oral administration, comprising N⁶-cyclopentyl-5′-(N-ethyl)carboxamidoadenosine or a pharmaceutically acceptable salt or ester thereof, and a pharmaceutically acceptable matrix adapted to provide a controlled release of N⁶-cyclopentyl-5′-(N-ethyl)carboxamidoadenosine (selodenoson) or a pharmaceutically acceptable salt or ester thereof upon oral administration, having a dissolution rate in vitro when measured using (i) Apparatus 3 described in the USP 23 using 300 mL reciprocating cylinder at 30 dips/minute in 250 mL 0.1 M hydrochloric acid from 0 minutes to 2 hours of dissolution and in 250 mL pH 6.8 phosphate buffer after 2 hours and up to 12 hours of dissolution, at 37.0±0.5° C. and using high performance liquid chromatography (HPLC) for quantification, or (ii) Apparatus 2 (paddle) described in USP 23 at 50 rpm in 500 mL 0.1 M hydrochloric acid or 0.005% SDS-0.1 M hydrochloric acid from 0 minutes to 2 hours of dissolution and in 900 mL pH 6.8 phosphate buffer, or pH 6.8 phosphate buffer containing 0.5% SDS, after 2 hours and up to 12 hours of dissolution, at 37.0±0.5° C. and using high performance liquid chromatography (HPLC) for quantification:
between 0 and 35% selodenoson released after 15 minutes;
between 0 and 50% selodenoson released after 30 minutes;
between 0.5 and 60% selodenoson released after 45 minutes;
between 0.5 and 65% selodenoson released after 1 hour;
between 0.5 and 90% selodenoson released after 2 hours;
between 20 and 90% selodenoson released after 4 hours;
between 25 and 100% selodenoson released after 8 hours; and
greater than 30% selodenoson released after 12 hours, by weight.

2. The formulation of claim 1, having a dissolution rate in vitro when measured using (i) Apparatus 3 described in the USP 23 using 300 mL reciprocating cylinder at 30 dips/minute in 250 mL 0.1 M hydrochloric acid from 0 minutes to 2 hours of dissolution and in 250 mL pH 6.8 phosphate buffer after 2 hours and up to 12 hours of dissolution, at 37.0±0.5° C. and using high performance liquid chromatography (HPLC) for quantification, or (ii) Apparatus 2 (paddle) described in USP 23 at 50 rpm in 500 mL 0.1 M hydrochloric acid or 0.005% SDS-0.1 M hydrochloric acid from 0 minutes to 2 hours of dissolution and in 900 m]L pH 6.8 phosphate buffer, or pH 6.8 phosphate buffer containing 0.5% SDS, after 2 hours and up to 12 hours of dissolution, at 37.0±0.5° C. and using high performance liquid chromatography (HPLC) for quantification:
between 0 and 35% selodenoson released after 15 minutes;
between 0.5 and 50% selodenoson released after 30 minutes;
between 0.5 and 60% selodenoson released after 45 minutes;
between 1 and 65% selodenoson released after 1 hour;
between 2 and 85% selodenoson released after 2 hours;
between 25 and 90% selodenoson released after 4 hours;
between 40 and 100% selodenoson released after 8 hours; and
greater than 55% selodenoson released after 12 hours, by weight.

3. The formulation of claim 1, having a dissolution rate in vitro when measured using (i) Apparatus 3 described in the USP 23 using 300 mL reciprocating cylinder at 30 dips/minute in 250 mL 0.1 M hydrochloric acid from 0 minutes to 2 hours of dissolution and in 250 mL pH 6.8 phosphate buffer after 2 hours and up to 12 hours of dissolution, at 37.0±0.5° C. and using high performance liquid chromatography (HPLC) for quantification, or (ii) Apparatus 2 (paddle) described in USP 23 at 50 rpm in 500 mL 0.1 M hydrochloric acid or 0.005% SDS-0.1 M hydrochloric acid from 0 minutes to 2 hours of dissolution and in 900 mL pH 6.8 phosphate buffer, or pH 6.8 phosphate buffer containing 0.5% SDS, after 2 hours and up to 12 hours of dissolution, at 37.0±0.5° C. and using high performance liquid chromatography (HPLC) for quantification:
between 0 and 35% selodenoson released after 15 minutes;
between 5 and 50% selodenoson released after 30 minutes;
between 10 and 60% selodenoson released after 45 minutes;
between 10 and 65% selodenoson released after 1 hour;
between 15 and 90% selodenoson released after 2 hours;
between 20 and 90% selodenoson released after 4 hours;
between 25 and 100% selodenoson released after 8 hours; and
greater than 30% selodenoson released after 12 hours, by weight.

4. The formulation of claim 3, having a dissolution rate in vitro when measured using (i) Apparatus 3 described in the USP 23 using 300 mL reciprocating cylinder at 30 dips/minute in 250 mL 0.1 M hydrochloric acid from 0 minutes to 2 hours of dissolution and in 250 mL pH 6.8 phosphate buffer after 2 hours and up to 12 hours of dissolution, at 37.0±0.5° C. and using high performance liquid chromatography (HPLC) for quantification, or (ii) Apparatus 2 (paddle) described in USP 23 at 50 rpm in 500 mL 0.1 M hydrochloric acid or 0.005% SDS-0.1 M hydrochloric acid from 0 minutes to 2 hours of dissolution and in 900 mL pH 6.8 phosphate buffer, or pH 6.8 phosphate buffer containing 0.5% SDS, after 2 hours and up to 12 hours of dissolution, at 37.0±0.5° C. and using high performance liquid chromatography (HPLC) for quantification:
between 0 and 35% selodenoson released after 15 minutes;
between 5 and 50% selodenoson released after 30 minutes;
between 10 and 60% selodenoson released after 45 minutes;
between 15 and 65% selodenoson released after 1 hour;
between 25 and 85% selodenoson released after 2 hours;
between 35 and 90% selodenoson released after 4 hours;
between 45 and 100% selodenoson released after 8 hours; and
greater than 55% selodenoson released after 12 hours, by weight.

5. The formulation of claim 3, having a dissolution rate in vitro when measured using Apparatus 3 described in the USP 23 using 300 mL reciprocating cylinder at 30 dips/minute in 250 mL 0.1 M hydrochloric acid at 0 minutes to 2 hours of dissolution and in 250 mL pH 6.8 phosphate buffer after 2 hours and up to 12 hours of dissolution at 37.0±0.5° C. and using high performance liquid chromatography (HPLC) for quantification:

| | % selodenoson |
|---|---|
| Time/min. | released |
| 15 | 0-20 |
| 30 | 5-30 |
| 45 | 10-40 |
| 60 | 10-45 |
| 120 | 15-60 |
| 240 | 20-65 |
| 480 | 25-85 |
| 720 | 30-100 |

6. The formulation of claim 5, having a dissolution rate in vitro when measured using Apparatus 3 described in the USP 23 using 300 mL reciprocating cylinder at 30 dips/minute in 250 mL 0 mL 0.1 M hydrochloric acid at 0 minutes to 2 hours of dissolution and in 250 mL pH 6.8 phosphate buffer after 2 hours and up to 12 hours of dissolution at 37.0±0.5° C. and using high performance liquid chromatography (HPLC) for quantification:

| | % selodenoson |
|---|---|
| Time/min. | released |
| 15 | 0-20 |
| 30 | 10-30 |
| 45 | 15-40 |
| 60 | 20-45 |
| 120 | 30-60 |
| 240 | 40-65 |
| 480 | 45-85 |
| 720 | 60-100 |

7. The formulation of claim 4, having a dissolution rate in vitro when measured using Apparatus 3 described in the USP 23 using 300 mL reciprocating cylinder at 30 dips/minute in 250 mL 0 mL 0.1 M hydrochloric acid at 0 minutes to 2 hours of dissolution and in 250 mL pH 6.8 phosphate buffer after 2 hours and up to 12 hours of dissolution at 37.0±0.5° C. and using high performance liquid chromatography (HPLC) for quantification:
between 5 and 20% by weight selodenoson released after 15 minutes,
between 10 and 30% by weight selodenoson released after 30 minutes,
between 15 and 35% by weight selodenoson released after 45 minutes,
between 20 and 40% by weight selodenoson released after 1 hour,
between 30 and 55% by weight selodenoson released after 2 hours,
between 40 and 65% by weight selodenoson released after 4 hours,
between 45 and 75% by weight selodenoson released after 8 hours, and
between 60 and 85% by weight selodenoson released after 12 hours.

8. The formulation of claim 4, having a dissolution rate in vitro when measured using Apparatus 3 described in the USP 23 using 300 mL reciprocating cylinder at 30 dips/minute in 250 mL 0.1 M hydrochloric acid at 0 minutes to 2 hours of dissolution and in 250 mL pH 6.8 phosphate buffer after 2 hours and up to 12 hours of dissolution at 37.0±0.5° C. and using high performance liquid chromatography (HPLC) for quantification:
between 10 and 20% by weight selodenoson released after 15 minutes,
between 15 and 30% by weight selodenoson released after 30 minutes,
between 20 and 40% by weight selodenoson released after 45 minutes,
between 25 and 45% by weight selodenoson released after 1 hour,
between 40 and 60% by weight selodenoson released after 2 hours,
between 45 and 65% by weight selodenoson released after 4 hours,
between 60 and 80% by weight selodenoson released after 8 hours, and
between 75 and 90% by weight selodenoson released after 12 hours.

9. The formulation of claim 5, which is in the form of a hydrophobic tablet.

10. The formulation of claim 4, having a dissolution rate in vitro when measured using Apparatus 3 described in the USP 23 using 300 mL reciprocating cylinder at 30 dips/minute in 250 mL 0.1 M hydrochloric acid at 0 minutes to 2 hours of dissolution and in 250 mL pH 6.8 phosphate buffer after 2 hours and up to 12 hours of dissolution at 37.0±0.5° C. and using high performance liquid chromatography (HPLC) for quantification:

| | % selodenoson |
|---|---|
| Time/min. | released |
| 15 | 0-15 |
| 30 | 5-20 |
| 45 | 10-25 |
| 60 | 15-30 |
| 120 | 25-45 |
| 240 | 35-55 |
| 480 | 50-70 |
| 720 | 70-95 |

11. The formulation of claim 4, having a dissolution rate in vitro when measured using Apparatus 3 described in the USP 23 using 300 mL reciprocating cylinder at 30 dips/minute in 250 mL 0 mL 0.1 M hydrochloric acid at 0 minutes to 2 hours of dissolution and in 250 mL pH 6.8 phosphate buffer after 2 hours and up to 12 hours of dissolution at 37.0±0.5° C. and using high performance liquid chromatography (HPLC) for quantification:
between 5 and 15% weight selodenoson released after 15 minutes,
between 10 and 20% weight selodenoson released after 30 minutes,
between 15 and 30% weight selodenoson released after 45 minutes,
between 15 and 30% weight selodenoson released after 1 hour,
between 30 and 45% weight selodenoson released after 2 hours,
between 40 and 55% weight selodenoson released after 4 hours,
between 55 and 70% weight selodenoson released after 8 hours, and
between 70 and 95% weight selodenoson released after 12 hours.

12. The formulation of claim 4, having a dissolution rate in vitro when measured using Apparatus 3 described in the USP 23 using 300 mL reciprocating cylinder at 30 dips/minute in 250 mL 0 mL 0.1 M hydrochloric acid at 0 minutes to 2 hours of dissolution and in 250 mL pH 6.8 phosphate buffer after 2 hours and up to 12 hours of dissolution at 37.0±0.5° C. and using high performance liquid chromatography (HPLC) for quantification:
between 0 and 15% weight selodenoson released after 15 minutes,
between 5 and 20% weight selodenoson released after 30 minutes,
between 10 and 20% weight selodenoson released after 45 minutes,
between 15 and 25% weight selodenoson released after 1 hour,
between 25 and 40% weight selodenoson released after 2 hours,
between 35 and 50% weight selodenoson released after 4 hours,
between 50 and 65% weight selodenoson released after 8 hours, and
between 75 and 90% weight selodenoson released after 12 hours.

13. The formulation of claim 10, which is in the form of a hydrophilic tablet.

14. The formulation of claim 3, having a dissolution rate in vitro when measured using Apparatus 2 (paddle) described in USP 23 at 50 rpm in 500 mL 0.1 M hydrochloric acid or 0.005% SDS-0. 1 M hydrochloric acid from 0 minutes to 2 hours of dissolution and in 900 mL pH 6.8 phosphate buffer, or pH 6.8 phosphate buffer containing 0.5% SDS, after 2 hours and up to 12 hours of dissolution, at 37.0±0.5° C. and using high performance liquid chromatography (HPLC) for quantification:

| | % selodenoson |
|---|---|
| Time/min. | released |
| 15 | 5-35 |
| 30 | 10-50 |
| 45 | 20-60 |
| 60 | 25-65 |
| 120 | 35-90 |
| 240 | 55-90 |
| 480 | 60-100 |
| 720 | 60-100 |

15. The formulation of claim 4, having a dissolution rate in vitro when measured using Apparatus 2 (paddle) described in USP 23 at 50 rpm in 500 mL 0.1 M hydrochloric acid from 0 minutes to 2 hours of dissolution and in 900 mL pH 6.8 phosphate buffer after 2 hours and up to 12 hours of dissolution, at 37.0±0.5° C. and using high performance liquid chromatography (HPLC) for quantification:
between 10 and 35% by weight selodenoson released after 15 minutes,
between 20 and 50% by weight selodenoson released after 30 minutes,
between 25 and 60% by weight selodenoson released after 45 minutes,
between 30 and 65% by weight selodenoson released after 1 hour,
between 45 and 90% by weight selodenoson released after 2 hours,
between 55 and 90% by weight selodenoson released after 4 hours,
between 60 and 95% by weight selodenoson released after 8 hours, and
between 65 and 100% by weight selodenoson released after 12 hours.

16. The formulation of claim 4, having a dissolution rate in vitro when measured using Apparatus 2 (paddle) described in USP 23 at 50 rpm in 500 mL 0.005% SDS-0. 1 M hydrochloric acid from 0 minutes to 2 hours of dissolution and in 900 mL pH 6.8 phosphate buffer containing 0.5% SDS, after 2 hours and up to 12 hours of dissolution, at 37.0±0.5° C. and using high performance liquid chromatography (HPLC) for quantification:
between 5 and 20% by weight selodenoson released after 15 minutes,
between 15 and 30% by weight selodenoson released after 30 minutes,
between 20 and 40% by weight selodenoson released after 45 minutes,
between 25 and 50% by weight selodenoson released after 1 hour,
between 35 and 70% by weight selodenoson released after 2 hours,
between 60 and 90% by weight selodenoson released after 4 hours,
between 70 and 100% by weight selodenoson released after 8 hours, and
between 80 and 100% by weight selodenoson released after 12 hours.

17. The formulation of claim 14, which is in the form of a capsule filled with a plurality of controlled release pellets (spheroids).

18. The formulation of claim 1, having a dissolution rate in vitro when measured using Apparatus 2 (paddle) described in USP 23 at 50 rpm in 500 mL 0.005% SDS-0. 1 M hydrochloric acid from 0 minutes to 2 hours of dissolution and in 900 mL pH 6.8 phosphate buffer containing 0.5% SDS, after 2 hours and up to 12 hours of dissolution, at 37.0±0.5° C. and using high performance liquid chromatography (HPLC) for quantification:
between 0 and 5% by weight selodenoson released after 15 minutes,
between 0 and 5% by weight selodenoson released after 30 minutes,
between 0.5 and 6% by weight selodenoson released after 45 minutes,
between 0.5 and 6% by weight selodenoson released after 1 hour,
between 0.5 and 20% by weight selodenoson released after 2 hours,
between 20 and 65% by weight selodenoson released after 4 hours,
between 30 and 80% by weight selodenoson released after 8 hours, and
between 50 and 100% by weight selodenoson released after 12 hours.

19. The formulation of claim 18, having a dissolution rate in vitro when measured using Apparatus 2 (paddle) described in USP 23 at 50 rpm in 500 mL 0.005% SDS-0.1 M hydrochloric acid from 0 minutes to 2 hours of dissolution and in 900 mL pH 6.8 phosphate buffer containing 0.5% SDS, after 2 hours and up to 12 hours of dissolution, at 37.0±0.5° C. and using high performance liquid chromatography (HPLC) for quantification:
between 0 and 3% by weight selodenoson released after 15 minutes,
between 0.5 and 3% by weight selodenoson released after 30 minutes,
between 0.5 and 5% by weight selodenoson released after 45 minutes,
between 1 and 5% by weight selodenoson released after 1 hour,
between 2 and 15% by weight selodenoson released after 2 hours,
between 25 and 60% by weight selodenoson released after 4 hours,
between 40 and 75% by weight selodenoson released after 8 hours, and
between 60 and 90% by weight selodenoson released after 12 hours.

20. The formulation of claim 18, which is in the form of a capsule filled with a plurality of controlled release pellets (spheroids) coated with a controlled release film coat.

21. The formulation of claim 1, wherein said formulation is suitable for dosing every 12 hours or more.

22. The formulation of claim 21, wherein said formulation is suitable for dosing every 24 hours.

23. The formulation of claim 1, wherein said formulation comprises from about 0.1 mg to about 50 mg of N⁶-cyclopentyl-5′-(N-ethyl)carboxamidoadenosine or a pharmaceutically acceptable salt or ester thereof.

24. The formulation of claim 23, wherein said formulation comprises from about 1 mg to about 20 mg of N⁶-cyclopentyl-5′-(N-ethyl)carboxamidoadenosine or a pharmaceutically acceptable salt or ester thereof.

25. The formulation of claim 1, wherein said formulation comprises from about 3 mg to about 10 mg of N⁶-cyclopentyl-5′-(N-ethyl)carboxamidoadenosine or a pharmaceutically acceptable salt or ester thereof.

26. The formulation of claim 1, wherein said formulation comprises from about 1 mg to about 3 mg of N⁶-cyclopentyl-5′-(N-ethyl)carboxamidoadenosine or a pharmaceutically acceptable salt or ester thereof.

27. The formulation claim 1, wherein said formulation is in the form of a tablet, a capsule, a sachet, or a plurality of multiparticulates.

28. The formulation of claim 27, wherein said plurality of multiparticulates comprises a plurality of granules, spheroids, or pellets.

29. A controlled release oral formulation suitable for dosing every 24 hours, comprising
a substrate comprising a pharmaceutically effective amount of N⁶-cyclopentyl-5′-(N-ethyl)carboxamidoadenosine (selodenoson) or a pharmaceutically acceptable salt or ester thereof; and
said substrate being incorporated in a controlled release matrix;
said formulation having a dissolution rate in vitro when measured using (i) Apparatus 3 described in the USP 23 using 300 mL reciprocating cylinder at 30 dips/minute in 250 mL 0.1 M hydrochloric acid from 0 minutes to 2 hours of dissolution and in 250 mL pH 6.8 phosphate buffer after 2 hours and up to 12 hours of dissolution, at 37.0±0.5° C. and using high performance liquid chromatography (HPLC) for quantification or (ii) Apparatus 2 (paddle) described in USP 23 at 50 rpm in 500 mL 0.1 M hydrochloric acid or 0.005% SDS-0. 1 M hydrochloric acid from 0 minutes to 2 hours of dissolution and in 900 mL pH 6.8 phosphate buffer, or pH 6.8 phosphate buffer containing 0.5% SDS, after 2 hours and up to 12 hours of dissolution, at 37.0±0.5° C. and using high performance liquid chromatography (HPLC) for quantification:
between 0 and 35% selodenoson released after 15 minutes;
between 0 and 50% selodenoson released after 30 minutes;
between 0.5 and 60% selodenoson released after 45 minutes;
between 0.5 and 65% selodenoson released after 1 hour;
between 0.5 and 90% selodenoson released after 2 hours;
between 20 and 90% selodenoson released after 4 hours;
between 25 and 100% selodenoson released after 8 hours; and
greater than 30% selodenoson released after 12 hours, by weight,
said formulation providing a therapeutic effect for about 24 hours after oral administration.

30. The formulation of claim 29, said formulation having a dissolution rate in vitro when measured using (i) Apparatus 3 described in the USP 23 using 300 1L reciprocating cylinder at 30 dips/minute in 250 mL 0.1 M hydrochloric acid from 0 minutes to 2 hours of dissolution and in 250 mL pH 6.8 phosphate buffer after 2 hours and up to 12 hours of dissolution, at 37.0±0.5° C. and using high performance liquid chromatography (HPLC) for quantification or (ii) Apparatus 2 (paddle) described in USP 23 at 50 rpm in 500 mL 0.1 M hydrochloric acid or 0.005% SDS-0.1 M hydrochloric acid from 0 minutes to 2 hours of dissolution and in 900 mL pH 6.8 phosphate buffer, or pH 6.8 phosphate buffer containing 0.5% SDS, after 2 hours and up to 12 hours of dissolution, at 37.0±0.5° C. and using high performance liquid chromatography (HPLC) for quantification:
between 0 and 35% selodenoson released after 15 minutes;
between 0.5 and 50% selodenoson released after 30 minutes;
between 0.5 and 60% selodenoson released after 45 minutes;
between 1 and 65% selodenoson released after 1 hour;
between 2 and 85% selodenoson released after 2 hours;
between 25 and 90% selodenoson released after 4 hours;
between 40 and 100% selodenoson released after 8 hours; and
greater than 55% selodenoson released after 12 hours, by weight,
said formulation providing a therapeutic effect for about 24 hours after oral administration.

31. The formulation of claim 29, said formulation having a dissolution rate in vitro when measured using (i) Apparatus 3 described in the USP 23 using 300 mL reciprocating cylinder at 30 dips/minute in 250 mL 0.1 M hydrochloric acid from 0 minutes to 2 hours of dissolution and in 250 mL pH 6.8 phosphate buffer after 2 hours and up to 12 hours of dissolution, at 37.0±0.5° C. and using high performance liquid chromatography (HPLC) for quantification or (ii) Apparatus 2 (paddle) described in USP 23 at 50 rpm in 500 mL 0.1 M hydrochloric acid or 0.005% SDS-0.1 M hydrochloric acid from 0 minutes to 2 hours of dissolution and in 900 mL pH 6.8 phosphate buffer, or pH 6.8 phosphate buffer containing 0.5% SDS, after 2 hours and up to 12 hours of dissolution, at 37.0±0.5° C. and using high performance liquid chromatography (HPLC) for quantification:
between 0 and 35% selodenoson released after 15 minutes;
between 5 and 50% selodenoson released after 30 minutes;
between 10 and 60% selodenoson released after 45 minutes;
between 10 and 65% selodenoson released after 1 hour;
between 15 and 90% selodenoson released after 2 hours;
between 20 and 90% selodenoson released after 4 hours;
between 25 and 100% selodenoson released after 8 hours; and
greater than 30% selodenoson released after 12 hours, by weight,
said formulation providing a therapeutic effect for about 24 hours after oral administration.

32. The formulation of claim 31, said formulation having a dissolution rate in vitro when measured using (i) Apparatus 3 described in the USP 23 using 300 mL reciprocating cylinder at 30 dips/minute in 250 mL 0.1 M hydrochloric acid from 0 minutes to 2 hours of dissolution and in 250 mL pH 6.8 phosphate buffer after 2 hours and up to 12 hours of dissolution, at 37.0±0.5° C. and using high performance liquid chromatography (HPLC) for quantification or (ii) Apparatus 2 (paddle) described in USP 23 at 50 rpm in 500 mL 0.1 M hydrochloric acid or 0.005% SDS-0.1 M hydrochloric acid from 0 minutes to 2 hours of dissolution and in 900 mL pH 6.8 phosphate buffer, or pH 6.8 phosphate buffer containing 0.5% SDS, after 2 hours and up to 12 hours of dissolution, at 37.0±0.5° C. and using high performance liquid chromatography (HPLC) for quantification:
between 0 and 35% selodenoson released after 15 minutes;
between 5 and 50% selodenoson released after 30 minutes;
between 10 and 60% selodenoson released after 45 minutes;
between 15 and 65% selodenoson released after 1 hour;
between 25 and 85% selodenoson released after 2 hours;
between 35 and 90% selodenoson released after 4 hours;
between 45 and 100% selodenoson released after 8 hours; and
greater than 55% selodenoson released after 12 hours, by weight,
said formulation providing a therapeutic effect for about 24 hours after oral administration.

33. The formulation of claim 31, having a dissolution rate in vitro when measured using Apparatus 3 described in the USP 23 using 300 mL reciprocating cylinder at 30 dips/minute in 250 mL 0.1 M hydrochloric acid at 0 minutes to 2 hours of dissolution and in 250 mL pH 6.8 phosphate buffer after 2 hours and up to 12 hours of dissolution at 37.0±0.5° C. and using high performance liquid chromatography (HPLC) for quantification:

| | % selodenoson |
|---|---|
| Time/min. | released |
| 15 | 0-20 |
| 30 | 5-30 |
| 45 | 10-40 |
| 60 | 10-45 |
| 120 | 15-60 |
| 240 | 20-65 |
| 480 | 25-85 |
| 720 | 30-100 |

34. The formulation of claim 33, having a dissolution rate in vitro when measured using Apparatus 3 described in the USP 23 using 300 mL reciprocating cylinder at 30 dips/minute in 250 mL 0 mL 0.1 M hydrochloric acid at 0 minutes to 2 hours of dissolution and in 250 mL pH 6.8 phosphate buffer after 2 hours and up to 12 hours of dissolution at 37.0±0.5° C. and using high performance liquid chromatography (HPLC) for quantification:

| | % selodenoson |
|---|---|
| Time/min. | released |
| 15 | 0-20 |
| 30 | 10-30 |
| 45 | 15-40 |
| 60 | 20-45 |
| 120 | 30-60 |
| 240 | 40-65 |
| 480 | 45-85 |
| 720 | 60-100 |

35. The formulation of claim 32, having a dissolution rate in vitro when measured using Apparatus 3 described in the USP 23 using 300 mL reciprocating cylinder at 30 dips/minute in 250 mL 0 mL 0.1 M hydrochloric acid at 0 minutes to 2 hours of dissolution and in 250 mL pH 6.8 phosphate buffer after 2 hours and up to 12 hours of dissolution at 37.0±0.5° C. and using high performance liquid chromatography (HPLC) for quantification:
between 5 and 20% by weight selodenoson released after 15 minutes,
between 10 and 30% by weight selodenoson released after 30 minutes,
between 15 and 35% by weight selodenoson released after 45 minutes,
between 20 and 40% by weight selodenoson released after 1 hour,
between 30 and 55% by weight selodenoson released after 2 hours,
between 40 and 65% by weight selodenoson released after 4 hours,
between 45 and 75% by weight selodenoson released after 8 hours, and
between 60 and 85% by weight selodenoson released after 12 hours.

36. The formulation of claim 32, having a dissolution rate in vitro when measured using Apparatus 3 described in the USP 23 using 300 mL reciprocating cylinder at 30 dips/minute in 250 mL 0.1 M hydrochloric acid at 0 minutes to 2 hours of dissolution and in 250 mL pH 6.8 phosphate buffer after 2 hours and up to 12 hours of dissolution at 37.0±0.5° C. and using high performance liquid chromatography (HPLC) for quantification:
between 10 and 20% by weight selodenoson released after 15 minutes,
between 15 and 30% by weight selodenoson released after 30 minutes,
between 20 and 40% by weight selodenoson released after 45 minutes,
between 25 and 45% by weight selodenoson released after 1 hour,
between 40 and 60% by weight selodenoson released after 2 hours,
between 45 and 65% by weight selodenoson released after 4 hours,
between 60 and 80% by weight selodenoson released after 8 hours, and
between 75 and 90% by weight selodenoson released after 12 hours.

37. The formulation of claim 32, having a dissolution rate in vitro when measured using Apparatus 3 described in the USP 23 using 300 mL reciprocating cylinder at 30 dips/minute in 250 mL 0.1 M hydrochloric acid at 0 minutes to 2 hours of dissolution and in 250 mL pH 6.8 phosphate buffer after 2 hours and up to 12 hours of dissolution at 37.0±0.5° C. and using high performance liquid chromatography (HPLC) for quantification:

| | % selodenoson |
|---|---|
| Time/min. | released |
| 15 | 0-15 |
| 30 | 5-20 |
| 45 | 10-25 |
| 60 | 15-30 |
| 120 | 25-45 |
| 240 | 35-55 |
| 480 | 50-70 |
| 720 | 70-95 |

38. The formulation of claim 32, having a dissolution rate in vitro when measured using Apparatus 3 described in the USP 23 using 300 mL reciprocating cylinder at 30 dips/minute in 250 mL 0 mL 0.1 M hydrochloric acid at 0 minutes to 2 hours of dissolution and in 250 mL pH 6.8 phosphate buffer after 2 hours and up to 12 hours of dissolution at 37.0≅0.5° C. and using high performance liquid chromatography (HPLC) for quantification:
between 5 and 15% by weight selodenoson released after 15 minutes,
between 10 and 20% by weight selodenoson released after 30 minutes,
between 15 and 30% by weight selodenoson released after 45 minutes,
between 15 and 30% by weight selodenoson released after 1 hour,
between 30 and 45% by weight selodenoson released after 2 hours,
between and 40 and 55% by weight selodenoson released after 4 hours,
between 55 and 70% by weight selodenoson released after 8 hours, and
between 70 and 95% by weight selodenoson released after 12 hours.

39. The formulation of claim 32, having a dissolution rate in vitro when measured using Apparatus 3 described in the USP 23 using 300 mL reciprocating cylinder at 30 dips/minute in 250 mL 0 mL 0.1 M hydrochloric acid at 0 minutes to 2 hours of dissolution and in 250 mL pH 6.8 phosphate buffer after 2 hours and up to 12 hours of dissolution at 37.0±0.5° C. and using high performance liquid chromatography (HPLC) for quantification:
between 0 and 15% by weight selodenoson released after 15 minutes,
between 5 and 20% by weight selodenoson released after 30 minutes,
between 10 and 20% by weight selodenoson released after 45 minutes,
between 15 and 25% by weight selodenoson released after 1 hour,
between 25 and 40% by weight selodenoson released after 2 hours,
between 35 and 50% by weight selodenoson released after 4 hours,
between 50 and 65% by weight selodenoson released after 8 hours, and
between 75 and 90% by weight selodenoson released after 12 hours.

40. The formulation of claim 31, having a dissolution rate in vitro when measured using Apparatus 2 (paddle) described in USP 23 at 50 rpm in 500 mL 0.1 M hydrochloric acid or 0.005% SDS-0.1 M hydrochloric acid from 0 minutes to 2 hours of dissolution and in 900 mL pH 6.8 phosphate buffer, or pH 6.8 phosphate buffer containing 0.5% SDS, after 2 hours and up to 12 hours of dissolution, at 37.0±0.5° C. and using high performance liquid chromatography (HPLC) for quantification:

| | % selodenoson |
|---|---|
| Time/min. | released |
| 15 | 5-35 |
| 30 | 10-50 |
| 45 | 20-60 |
| 60 | 25-65 |
| 120 | 35-90 |
| 240 | 55-90 |
| 480 | 60-100 |
| 720 | 60-100 |

41. The formulation of claim 32, having a dissolution rate in vitro when measured sing Apparatus 2 (paddle) described in USP 23 at 50 rpm in 500 mL 0.1 M hydrochloric acid from 0 minutes to 2 hours of dissolution and in 900 mL pH 6.8 phosphate buffer after 2 hours and up to 12 hours of dissolution, at 37.0±0.5° C. and using high performance liquid chromatography (HPLC) for quantification:
between 10 and 35% by weight selodenoson released after 15 minutes,
between 20 and 50% by weight selodenoson released after 30 minutes,
between 25 and 60% by weight selodenoson released after 45 minutes,
between 30 and 65% by weight selodenoson released after 1 hour,
between 45 and 90% by weight selodenoson released after 2 hours,
between 55 and 90% by weight selodenoson released after 4 hours,
between 60 and 95% by weight selodenoson released after 8 hours, and
between 65 and 100% by weight selodenoson released after 12 hours.

42. The formulation of claim 32, having a dissolution rate in vitro when measured using Apparatus 2 (paddle) described in USP 23 at 50 rpm in 500 mL 0.005% SDS-0.1 M hydrochloric acid from 0 minutes to 2 hours of dissolution and in 900 mL pH 6.8 phosphate buffer containing 0.5% SDS, after 2 hours and up to 12 hours of dissolution, at 37.0±0.5° C. and using high performance liquid chromatography (HPLC) for quantification:
between 5 and 20% by weight selodenoson released after 15 minutes,
between 15 and 30% by weight selodenoson released after 30 minutes,
between 20 and 40% by weight selodenoson released after 45 minutes,
between 25 and 50% by weight selodenoson released after 1 hour,
between 35 and 70% by weight selodenoson released after 2 hours,
between 60 and 90% by weight selodenoson released after 4 hours,
between 70 and 100% by weight selodenoson released after 8 hours, and
between 80 and 100% by weight selodenoson released after 12 hours.

43. The formulation of claim 29, having a dissolution rate in vitro when measured using Apparatus 2 (paddle) described in USP 23 at 50 rpm in 500 mL 0.005% SDS-0.1 M hydrochloric acid from 0 minutes to 2 hours of dissolution and in 900 mL pH 6.8 phosphate buffer containing 0.5% SDS, after 2 hours and up to 12 hours of dissolution, at 37.0±0.5° C. and using high performance liquid chromatography (HPLC) for quantification:
between 0 and 5% by weight selodenoson released after 15 minutes,
between 0 and 5% by weight selodenoson released after 30 minutes,
between 0.5 and 6% by weight selodenoson released after 45 minutes,
between 0.5 and 6% by weight selodenoson released after 1 hour,
between 0.5 and 20% by weight selodenoson released after 2 hours,
between 20 and 65% by weight selodenoson released after 4 hours,
between 30 and 80% by weight selodenoson released after 8 hours, and
between 50 and 100% by weight selodenoson released after 12 hours.

44. The formulation of claim 43, having a dissolution rate in vitro when measured using Apparatus 2 (paddle) described in USP 23 at 50 rpm in 500 mL 0.005% SDS-0.1 M hydrochloric acid from 0 minutes to 2 hours of dissolution and in 900 mL pH 6.8 phosphate buffer containing 0.5% SDS, after 2 hours and up to 12 hours of dissolution, at 37.0±0.5° C. and using high performance liquid chromatography (HPLC) for quantification:
between 0 and 3% by weight selodenoson released after 15 minutes,
between 0.5 and 3% by weight selodenoson released after 30 minutes,
between 0.5 and 5% by weight selodenoson released after 45 minutes,
between 1 and 5% by weight selodenoson released after 1 hour,
between 2 and 15% by weight selodenoson released after 2 hours,
between 25 and 60% by weight selodenoson released after 4 hours,
between 40 and 75% by weight selodenoson released after 8 hours, and
between 60 and 90% by weight selodenoson released after 12 hours.

45. The formulation of claim 29, wherein said formulation is a tablet.

46. The formulation of claim 29, wherein said formulation is a capsule filled with a plurality of multiparticulates.

47. The formulation of claim 46, wherein said formulation is a capsule filled with a plurality of spheroids.

48. The formulation of claim 1, wherein said matrix comprises at least one hydroxyalkylcellulose.

49. The formulation of claim 1, wherein said matrix comprises one or more hydrogenated vegetable oils, one or more glyceryl ester of a fatty acid or mixtures thereof.

50. The formulation of claim 1, wherein said matrix comprises a hydrophobic, fusible carrier or diluent and at least one digestible, long-chain hydrocarbon.

51. A solid, controlled release formulation suitable for oral administration, comprising a pharmaceutically effective amount of N⁶-cyclopentyl-5′-(N-ethyl)carboxamidoadenosine or a pharmaceutically acceptable salt or ester thereof, and a controlled release matrix.

52. The formulation of claim 51, wherein said matrix comprises at least one hydrophilic polymer.

53. The formulation of claim 52, wherein said matrix comprises one or more hydroxyalkylcellulose.

54. The formulation of claim 53, wherein said matrix comprises from about 30% to about 75% by weight of dosage unit of one or more hydroxyalkylcellulose.

55. The formulation of claim 54, wherein said matrix comprises from about 40% to about 70% by weight of dosage unit of one or more hydroxyalkylcellulose.

56. The formulation of claim 53, wherein said one or more hydroxyalkylcellulose is selected from the group consisting of hydroxypropylcellulose and hydroxypropylmethylcellulose.

57. The formulation of claim 51, wherein said matrix comprises at least one hydrophobic polymer.

58. The formulation of claim 51, wherein said matrix comprises one or more digestible, long-chain hydrocarbon.

59. The formulation of claim 58, wherein said matrix comprises one or more hydrogenated vegetable oil, one or more glyceryl ester of a fatty acid or mixtures thereof.

60. The formulation of claim 59, wherein said matrix comprises from about 10% to about 65% by weight of dosage unit of one or more hydrogenated vegetable oil and from about 10% to about 50% by weight of dosage unit of one or more glyceryl ester of a fatty acid.

61. The formulation of claim 60, wherein said matrix comprises from about 15% to about 45% by weight of dosage unit of one or more hydrogenated vegetable oil and from about 15% to about 45% by weight of dosage unit of one or more glyceryl ester of a fatty acid.

62. The formulation of claim 60, wherein said one or more glyceryl ester of a fatty acid comprises castor oil in an amount of from about 5% to about 25% by weight of dosage unit.

63. The formulation of claim 58, wherein said one or more digestible, long-chain hydrocarbon comprises castor oil in an amount of about 5-25% by weight of dosage unit.

64. The formulation of claim 51, wherein said matrix comprises a hydrophobic, fusible carrier or diluent and at least one digestible, long-chain hydrocarbon.

65. The formulation of claim 64, wherein the amount of said hydrophobic, fusible carrier or diluent is from about 3% to about 18% by weight of dosage unit and the amount of said one or more digestible, long-chain hydrocarbon is from about 35% to about 95% by weight of dosage unit.

66. The formulation of claim 51, further comprising one or more diluent.

67. The formulation of claim 66, wherein the amount of said one or more diluent is from about 5% to about 70% by weight of dosage unit.

68. The formulation of claim 67, wherein said amount is from about 10% to about 45% by weight of dosage unit.

69. The formulation of claim 51, further comprising a film coating comprising a controlled release coating material.

70-99. (canceled)

100. The formulation of claim 29, wherein said matrix comprises at least one hydroxyalkylcellulose.

101. The formulation of claim 29, wherein said matrix comprises one or more hydrogenated vegetable oils, one or more glyceryl ester of a fatty acid or mixtures thereof.

102. The formulation of claim 29, wherein said matrix comprises a hydrophobic, fusible carrier or diluent and at least one digestible, long-chain hydrocarbon.
